(19) United States
(12) Patent Application Publication
(10) Pub. No.: US 2021/0236445 A1
(43) Pub. Date: Aug. 5, 2021

(54) BACLOFEN AND ACAMPROSATE BASED THERAPY OF ALZHEIMER'S DISEASE IN PATIENTS HAVING LOST RESPONSIVENESS TO ACETYLCHOLINESTERASE INHIBITOR THERAPY

(71) Applicant: PHARNEXT, ISSY LES MOULINEAUX (FR)

(72) Inventors: ANTHONY BRUREAU, CHILLY-MAZARIN (FR); NATHALIE CHOLET, CLAMART (FR); DANIEL COHEN, SAINT CLOUD (FR); RODOLPHE HAJJ, SAINT GERMAIN EN LAYE (FR); SERGUEI NABIROCHKIN, CHATENAY MALABRY (FR)

(21) Appl. No.: 16/965,310

(22) PCT Filed: Jan. 28, 2019
(86) PCT No.: PCT/EP2019/051951

§ 371 Date: Jul. 28, 2020

(30) Foreign Application Priority Data
Jan. 29, 2018 (EP) 18305081.4
Jul. 20, 2018 (EP) 18184726.0

(51) Int. Cl. A61K 31/197; A61K 31/16; A61K 31/445; A61P 25/28

(52) U.S. Cl. CPC A61K 31/197; A61P 25/28; A61K 31/445; A61K 31/16

(57) ABSTRACT

The present invention relates to combinations and methods based on Baclofen and Acamprosate for the treatment of Alzheimer's disease or Alzheimer's related disorders in patients who do not respond to an inhibitor of acetylcholinesterase, typically in patients treated with an inhibitor of acetylcholinesterase and who have lost responsiveness to said inhibitor of acetylcholinesterase.

### CROSS-REFERENCE TO RELATED APPLICATION

This application is the U.S. national stage application of International Patent Application No. PCT/EP2019/051951, filed Jan. 28, 2019.

### FIELD OF THE INVENTION

The present invention relates to combinations and methods for the treatment of Alzheimer's disease or Alzheimer's related disorders in patients who do not respond to an inhibitor of acetylcholinesterase, typically in patients treated with an inhibitor of acetylcholinesterase and who have lost responsiveness to said inhibitor of acetylcholinesterase. More specifically, the present invention relates to novel combinatorial therapy, based on Baclofen and Acamprosate combination, for Alzheimer's or Alzheimer's related disorders patients already treated with an inhibitor of acetylcholinesterase and who have lost responsiveness to said inhibitor of acetylcholinesterase.

### BACKGROUND OF THE INVENTION

Alzheimer's disease (AD) is the prototypic cortical dementia characterized by memory deficit together with dysphasia (language disorder in which there is an impairment of speech and of comprehension of speech), dyspraxia (disability to coordinate and perform certain purposeful movements and gestures in the absence of motor or sensory impairments) and agnosia (ability to recognize objects, persons, sounds, shapes, or smells) attributable to involvement of the cortical association areas. Special symptoms such as spastic paraparesis (weakness affecting the lower extremities) can also be involved (1-4).

Incidence of Alzheimer disease increases dramatically with the age. AD is at present the most common cause of dementia. It is clinically characterized by a global decline of cognitive function that progresses slowly and leaves end-stage patients bound to bed, incontinent and dependent on custodial care. Death occurs, on average, 9 years after diagnosis (5).

The incidence rate of AD increases dramatically with age. United Nation population projections estimate that the number of people older than 80 years will approach 370 million by the year 2050. Currently, it is estimated that 50% of people older than age 85 years are afflicted with AD. Therefore, more than 100 million people worldwide will suffer from dementia in 50 years. The vast number of people requiring constant care and other services will severely affect medical, monetary and human resources (6).

Memory impairment is the early feature of the disease and involves episodic memory (memory for day-to-day events). Semantic memory (memory for verbal and visual meaning) is involved later in the disease. By contrast, working memory (short-term memory involving structures and processes used for temporarily storing and manipulating information) and procedural memory (unconscious memory that is long-term memory of skills and procedure) are preserved until late. As the disease progresses, the additional features of language impairment, visual perceptual and spatial deficits, agnosias and apraxias emerge.

The classic picture of Alzheimer's disease is sufficiently characteristic to allow identification in approximately 80% of cases (7). Nevertheless, clinical heterogeneity does occur and not only is this important for clinical management but provides further implication of specific medication treatments for functionally different forms (8).

The pathological hallmarks of AD include deposition of extracellular amyloid plaques containing beta-amyloid peptides (Abeta), intracellular neurofibrillary tangles (NFT) composed of Tau protein and progressive neuronal and synaptic dysfunction and loss (9-11). The etiology of AD remains elusive and for the last decades, several main hypotheses on the cause of AD have been proposed: the “cholinergic hypothesis” attributing a particular role for decrease acetylcholinergic signaling in development of AD, “amyloid cascade hypothesis”, which states that the neurodegenerative process is a series of events provoked by the abnormal processing of the Amyloid Precursor Protein (APP) (12), the revised “Tau hypothesis” (13), which proposes that cytoskeletal changes are the triggering pathological events, and recently, neuroimmomodulation hypothesis prioritizing changes in immune signaling in AD etiology and progression (14). The most widely accepted theory explaining AD progression remains the amyloid cascade hypothesis (15-17) and AD researchers have mainly focused on determining the mechanisms underlying the toxicity associated with amyloidogenic Abeta peptides. Importantly, changes in microvascular permeability and vessels remodeling, manifested as aberrant angiogenesis and blood brain barrier breakdown in course of AD, have been identified as key events implicated in the APP toxicity (18).

Synaptic density change is a pathological lesion that correlates better with cognitive impairment than the deposition of APP and Tau aggregates. Studies have revealed that the amyloid pathology appears to progress in a neurotransmitter-specific manner where the cholinergic terminals appear most vulnerable, followed by the glutamatergic terminals and finally by the GABAergic terminals (11). Glutamate is the most abundant excitatory neurotransmitter in the mammalian nervous system, and its functional effects are finely contra-balanced by GABAergic inhibitory neuronal receptors. Under pathological conditions, abnormal accumulation of glutamate in the synaptic cleft leads to glutamate receptors overactivation (19), that results in cognitive dysfunction and finally in neuronal cell death. This process, named excitotoxicity, is commonly observed in neuronal tissues during acute and chronic neurological disorders and is recognized now as one of the major pathological triggers in AD. Moreover, dysfunction in inhibitory GABA-mediated neuronal circuits observed in AD could increase negative consequences of dysregulated glutamate signaling in neuronal cells.

Patients diagnosed with mild-to-moderate AD are commonly treated with acetylcholinesterase inhibitors (19), such as Donepezil (DNPz), Galantamine, or Rivastigmine, considered standards of care. However, some patients do not respond to such therapy. Also, in responding patients, it appears the efficacy of acetylcholinesterase inhibitors decreases fast over time with disease progression, few months after initiation of treatment, leaving the patients without any treatment alternative. For example, studies on donepezil showed that the treatment improved the patient's cognitive function for the first 12 weeks, then the patient's cognitive function starts declining to reach its baseline level only 30 weeks after initiation of treatment (20, 21, 22 and 23). The same limited efficacy is described for rivastigmine (24) and galantamine (25). It results therefore that inhibitors of acetylcholinesterase only improve patients' cognitive functions during the first 12 weeks. After that period, the patients' cognitive functions start declining again. After only 6 months to 12 months of treatment, most patients will have regained the level of cognitive impairment suffered prior to treatment with their cognitive function further declining inexorably. Thus, such patients lose their responsiveness to acetylcholinesterase inhibitors.

WO2012/117076 discloses drug combinations for use in the treatment of AD, in particular the combination of baclofen and acamprosate. It also discloses that said combination can be further combined with existing treatment of AD such as donepezil, galantamine, rivastigmine and tacrine.

### SUMMARY OF INVENTION

It has now been found that baclofen and acamprosate may be used as an effective combination therapy of AD in patients who either do not respond to acetylcholinesterase inhibitors or have lost responsiveness to acetylcholinesterase inhibitors. The combination therapy is effective in such patients and can also restore responsiveness to acetylcholinesterase inhibitors.

It is thus an object of the present invention to provide new therapeutic methods and compositions for treating Alzheimer's disease in patients who do not respond to a treatment with an acetylcholinesterase inhibitor, comprising administering to the patient a combination of baclofen and acamprosate, or salts or derivatives thereof.

It is a further object of the present invention to provide methods and compositions for restoring responsiveness to a treatment with an acetylcholinesterase inhibitor in patients who do not respond to said treatment, comprising administering to the patient a combination of baclofen and acamprosate, or salts or derivatives thereof.

It is another object of the present invention to provide new therapeutic methods and compositions for treating Alzheimer's disease in patients treated with an inhibitor of acetylcholinesterase and who have lost responsiveness to said inhibitor of acetylcholinesterase, comprising administering to the patient a combination of baclofen and acamprosate, or salts or derivatives thereof.

The invention stems, inter alia, from the unexpected discovery, by the inventors, that the combination of Baclofen and Acamprosate provides substantial and unexpected benefit to patients with Alzheimer's disease under therapy with an inhibitor of acetylcholinesterase and who have lost optimal responsiveness to said inhibitor of acetylcholinesterase.

The invention also relates to compositions comprising Baclofen and Acamprosate, or pharmaceutically acceptable salts or derivatives thereof, for use in the treatment of Alzheimer's disease or an Alzheimer's disease related disorder in a subject not responding to an inhibitor of acetylcholinesterase.

A further object of this invention relates to compositions comprising a combination of Baclofen and Acamprosate, for use in the treatment of AD or an AD related disorder in patients suffering from such disease, wherein said patients are under treatment with an inhibitor of acetylcholinesterase and have lost responsiveness to said inhibitor of acetylcholinesterase.

Another object of this invention also relates to compositions comprising a combination of Baclofen and Acamprosate, for use in the treatment of AD or an AD related disorder in patients suffering from such disease, wherein said patients have been treated with an inhibitor of acetylcholinesterase for a period of at least 12 weeks, preferably 6 months, and have lost responsiveness to said inhibitor of acetylcholinesterase.

The composition of the invention may contain Baclofen and Acamprosate as the only active ingredients. Alternatively, the compositions may comprise additional active ingredient(s) such as, in particular, an inhibitor of acetylcholinesterase. In this regard, a further object of this invention relates to a composition comprising a combination of Baclofen, Acamprosate and an inhibitor of acetylcholinesterase for use in the treatment of AD and related disorders in a subject in need thereof, wherein said subject was initially under therapy with said inhibitor of acetylcholinesterase and has lost responsiveness to said acetylcholinesterase inhibitor. In a particular embodiment, the inhibitor of acetylcholinesterase is selected from donepezil, galantamine and rivastigmine. More particularly, the inhibitor of acetylcholinesterase is donepezil.

As it will be further disclosed in the present application, the compounds in a combinatorial therapy of the invention may be administered simultaneously, separately, sequentially and/or repeatedly to the subject.

The compositions of the invention typically further comprise one or several pharmaceutically acceptable excipients or carriers. Also, the compounds as used in the present invention may be in the form of a salt, hydrate, ester, ether, acid, amide, racemate, or isomer. They may also be in the form of sustained-release formulations. Prodrugs or derivatives of the compounds may be used as well.

In a preferred embodiment, the compound is used as such or in the form a salt, hydrate, ester, ether or sustained release form thereof. A particularly preferred salt for use in the present invention is Acamprosate calcium.

In another preferred embodiment, a prodrug or derivative is used.

The subject or patient may be any mammal, particularly a human, at any stage of the disease.

A preferred object of this invention relates to a method for treating Alzheimer disease in a human subject in need thereof, and wherein said subject is under treatment with an inhibitor of acetylcholinesterase and has lost responsiveness to said inhibitor of acetylcholinesterase, the method comprising simultaneously, separately or sequentially administering to said subject an effective amount of at least Baclofen and Acamprosate or salts or derivatives thereof. In a preferred embodiment, the method further comprises administering to the subject said inhibitor of acetylcholinesterase.

### BRIEF DESCRIPTION OF THE FIGURES

FIG. 1: Rescue effect of acamprosate and baclofen alone or as add-on therapy to declining donepezil on Aβ₂₅₋₃₅-induced spontaneous alternation deficits in mice. (A) Mice are injected ICV at Day 1 (D01) with amyloid β-peptide (25-35) or Scrambled.Aβ. Nineteen days later (D20), animals received the treatment (vehicle or donepezil (DNPz) (1 mg/Kg)) per os by gavage once a day (treatment with vehicle not represented in FIG. 1). At D30 one group with donepezil treatment was maintained on donepezil treatment alone (group 3), another group with donepezil treatment was administered, in addition to donepezil treatment, with acamprosate (ACP) and baclofen (BCL) (0.2 mg/Kg; 3 mg/Kg respectively) (group 4). At D30 and D38, animal cognitive performances in each group were tested by the Y-maze test. At Day D39/40 animal cognitive performances in each group were tested by Passive avoidance test. (B) Mice are injected ICV at Day 1 (D01) with amyloid β-peptide (25-35) or Scrambled.Aβ (Sc.Aβ). Nineteen days later (D20), animals received the treatment (vehicle, donepezil (DNPz) (1 mg/Kg) or a combination of acamprosate (ACP) and baclofen (BCL) (0.2 mg/Kg; 3 mg/Kg respectively)) per os by gavage once a day (treatment with vehicle not represented in FIG. 1). At D30 one group with donepezil treatment (1 mg/Kg) was maintained on donepezil treatment alone (group 5). In another group, treatment with donepezil (1 mg/Kg) was stopped at D30 and replaced with the administration of acamprosate and baclofen (0.2 mg/Kg; 3 mg/Kg respectively) (group 6). At last, at D30 one group with acamprosate and baclofen treatment (0.2 mg/Kg; 3 mg/Kg respectively) was maintained on acamprosate and baclofen treatment alone (group 7). At D30 and D40, animal cognitive performances in each group were tested by the Y-maze test. At Day D41/42, animal cognitive performances in each group were tested by Passive avoidance test.

FIG. 2: Rescue effect of acamprosate and baclofen alone or as add-on therapy to declining donepezil on Aβ₂₅₋₃₅-induced spontaneous alternation deficits in mice. (A) and (B) Spontaneous alternation performances assessed by Y-maze was performed at D30 after 11 days of treatment respectively for the groups tested according to FIG. 1. A protocol or FIG. 1.B protocol. (C) and (D) Spontaneous alternation performances assessed by Y-maze was performed at D38 after 19 days of treatment and D41 after 22 days of treatment respectively for the group of animals tested according to the protocol of FIG. 1.A. or FIG. 1.B. 1. Sc.Aβ injected animal group+vehicle treatment. 2. Aβ₂₅₋₃₅ injected animal group+vehicle treatment. 3 (Group 3). Aβ₂₅₋₃₅ injected animal group+Donepezil (1 mg/Kg) between D20 and D40. 4 (Group 4). Aβ₂₅₋₃₅ injected animal group+Donepezil (1 mg/Kg) between D20 and D40+acamprosate and baclofen (0.2 mg/Kg and 3 mg/Kg; respectively) between D30 to D40. 5 (Group 5). Aβ₂₅₋₃₅ injected animal group+Donepezil (1 mg/Kg) between D20 and D42. 6 (Group 6). Aβ₂₅₋₃₅ injected animal group +Donepezil (1 mg/Kg) between D20 and D29 and then acamprosate and baclofen (0.2 mg/Kg and 3 mg/Kg; respectively) between D30 to D42. 7 (Group 7). Aβ₂₅₋₃₅ injected animal group+acamprosate and baclofen (0.2 mg/Kg and 3 mg/Kg; respectively) between D20 and D42. Data are represented as mean and SEM. n=8 per group; *** p<0.001 vs. the Aβ₂₅₋₃₅/Veh group; ## p<0.01; ### p<0.001 vs. the Sc.Aβ/Veh group ANOVA followed by a Dunnett's test was performed.

FIG. 3: Rescue effect of acamprosate and baclofen alone or as add-on therapy to declining donepezil on Aβ₂₅₋₃₅-induced step-through passive avoidance deficits in mice. (A) and (B) Step-through latency assessed by passive avoidance test was performed at D40 after 21 days of treatment or D42 after 23 days of treatment respectively for the groups tested according to FIG. 1.A protocol or FIG. 1.B protocol. (C) and (D) Escape latency assessed by passive avoidance was performed at D40 after 21 days of treatment or D42 after 23 days of treatment respectively for the groups tested according to FIG. 1.A protocol or FIG. 1.B protocol. 1. Sc.Aβ injected animal group+vehicle treatment. 2. Aβ₂₅₋₃₅ injected animal group+vehicle treatment. 3. Aβ₂₅₋₃₅ injected animal group+Donepezil (1 mg/Kg) between D20 and D40. 4. Aβ₂₅₋₃₅ injected animal group+Donepezil (1 mg/Kg) between D20 and D40+acamprosate and baclofen (0.2 mg/Kg and 3 mg/Kg; respectively) between D30 to D40. 5. Aβ₂₅₋₃₅ injected animal group+Donepezil (1 mg/Kg) between D20 and D42. 6. Aβ₂₅₋₃₅ injected animal group+Donepezil (1 mg/Kg) between D20 and D29 and then acamprosate and baclofen (0.2 mg/Kg and 3 mg/Kg; respectively) between D30 to D42. 7. Aβ₂₅₋₃₅ injected animal group+acamprosate and baclofen (0.2 mg/Kg and 3 mg/Kg; respectively) between D20 and D42. Data are represented as mean and SEM. n=8 per group; *** p<0.001 vs. the Aβ₂₅₋₃₅/Veh group; # p<0.05; ## p<0.01; ### p<0.001 vs. the Sc.Aβ/Veh group Kruskall-Wallis followed by a Dunns test was performed.

FIG. 4: Three independent studies were used to demonstrate that donepezil efficacy declines when the treatment is initiated at later stages of the disease in an AD mouse model. Mice were intracerebroventricularly injected at Day 1 (D01) with amyloid β-peptide (25-35) or Scrambled.Aβ. Vehicle or donepezil were administered per os by gavage once a day starting at (A) D8 for a period of 10 days or (B) D20 for a period of 11 days or (C) D20 for a period of 21 days. At (A) D15, (B) D28, (C) D30 and D38, animal cognitive performances were tested by the Y-maze test. At (A) D16-17, (B) 29-30, (C) D39-40, animal cognitive performances were tested by passive avoidance test.

FIG. 5: Effect of DNPz on Aβ₂₅₋₃₅-induced cognitive deficits in mice at different timepoints of disease. 1. Sc.Aβ injected animal group+vehicle treatment. 2. Aβ₂₅₋₃₅ injected animal group+vehicle treatment. 3. Aβ₂₅₋₃₅ injected animal group+Donepezil between (A) D08 to D17, (B) D20 to D30 and (C) D20 to D40. At (A) D15, (B) D28, animal cognitive performances were tested by the Y-maze test, data are represented as mean and SEM. At (A) D16-17, (B) D29-30, animal cognitive performances were tested by passive avoidance test, data are represented as mean and SEM. (C) The animal cognitive performances were tested at D30 and D38, data are represented as mean of percentage drug effect and SEM. n is at least 8 per groups; *p<0,05; ** p<0,01; *** p<0.001 vs. the Aβ₂₅₋₃₅ Veh group; # p<0.05; ##<p<0.01; ### p<0.001 vs. the Sc.Aβ/Veh group ANOVA followed by a Dunnett's test was performed for spontaneous alternation performances, and Kruskall-Wallis followed by a Dunn's test was performed for passive avoidance test.

FIG. 6: (A) Donepezil effect declines when the treatment is initiated at later stages of the disease. Percentage drug effect of treatment periods of 10 days with Donepezil on Aβ₂₅₋₃₅-induced spontaneous alternation deficits in mice, assessed by Y-maze. (B) Percentage drug effect of treatment periods of 10 days with Donepezil on Aβ₂₅₋₃₅-induced cognitive impairments deficits in mice, assessed by passive avoidance test (step-through latency parameter). n is at least 8 per groups; data are represented as mean and SEM.

FIG. 7: Rescue effect of acamprosate and baclofen add-on therapy to declining donepezil on Aβ₂₅₋₃₅-induced spontaneous alternation deficits in mice. Mice were intracerebroventricularly injected at Day 1 (D01) with amyloid β-peptide (25-35) or Scrambled.Aβ. Six days later (D07) animals received the treatment (vehicle or donepezil (DNPz)(1 mg/Kg)) per os by gavage once a day. At D07, D14, D21, D28, D35, D41, and D48, cognitive performances are tested by the Y-maze test. In one group, when animals lost responsiveness to donepezil (D49), the treatment with donepezil was maintained until D100 (Group 3). In another group of animals, when animals lost responsiveness to donepezil (D49), the combination of acamprosate and baclofen (0.2 mg/Kg and 3 mg/Kg; respectively) was added. Cognitive performance was then tested by Y-maze at D56, D63, D70, D77, D91 and D98. At the end of the experiment (D99/D100), fear conditioning memory was assessed by Step-through passive avoidance (STPA).

FIG. 8: Rescue effect of acamprosate and baclofen add-on therapy to declining donepezil on Aβ₂₅₋₃₅-induced spontaneous alternation deficits in mice. (A) Evolution of spontaneous alternation assessed by Y-maze in animals between D7 and D100. (B) Step-through latency assessed by passive avoidance test was performed at D99/100.1. Sc.Aβ injected animal group+vehicle treatment. 2. Aβ₂₅₋₃₅ injected animal group +vehicle treatment. 3 (Group 3). Aβ₂₅₋₃₅ injected animal group+Donepezil (1 mg/Kg) 4 (Group 4). Aβ₂₅₋₃₅ injected animal group+Donepezil (1 mg/Kg) between D07 and D100, and supplemented with acamprosate and baclofen (0.2 mg/Kg and 3 mg/Kg; respectively) between D49 and D100. Data are represented as mean and SEM. n=8. ** p<0.01 vs. the Aβ₂₅₋₃₅/Veh group; ### p<0.001 vs. the Sc.Aβ/Veh group Kruskall-Wallis followed by a Dunn's test was performed for STL measurement.

### DETAILED DESCRIPTION OF THE INVENTION

It is an object of the present invention to provide new therapeutic methods and compositions for treating Alzheimer's disease (AD) or AD related disorders in subjects who do not respond (or have lost responsiveness) to treatment with an inhibitor of acetylcholinesterase.

The invention is suited for treating AD or AD related disorders such as any disorder characterized by dementia and/or associated with an abnormal processing and function of beta amyloid peptides. In the context of this invention, the term “AD related disorder” includes senile dementia of AD type (SDAT), frontotemporal dementia (FTD), vascular dementia, mild cognitive impairment (MCI) and age-associated memory impairment (AAMI), Parkinson's disease dementia, body Lewy dementia, mixed dementia, Creutzfeldt-Jakob disease, normal pressure hydrocephalus, Huntington's disease, Wernicke-Korsakoff syndrome, traumatic brain injury, progressive supranuclear palsy, corticobasal degeneration, down syndrome, Duchenne muscular dystrophy, multiple sclerosis.

As used herein, “treatment” includes the therapy, retardation or reduction of symptoms provoked by or of the causes of the above diseases or disorders. The term treatment includes in particular the control or reduction or reversion of disease progression and associated symptoms. The term treatment particularly includes a protection against the toxicity caused by beta amyloid (Abeta or Aβ are used interchangeably) peptides, or a reduction or retardation of said toxicity, in the treated subjects. The term treatment also designates an improvement of cognitive function or symptom, or a protection of neuronal cells.

As used herein, the term “non/not-responding” or “having lost responsiveness” to treatment with an acetylcholinesterase inhibitor, more particularly with an acetylcholinesterase inhibitor selected from the list consisting of donepezil, galantamine and rivastigmine, refers to a complete or partial lack of response to said inhibitor. A complete lack of response indicates that the inhibitor does not cause any benefit to the patient, particularly any cognitive benefit. A partial lack of response indicates that the inhibitor produces a suboptimal effect/benefit in the patient, particularly a suboptimal cognitive benefit. Partial means any incomplete effect, from 95% to 1% of the optimal response, typically less than 70%, such as less than 60%, or less than 50% of the optimal response observed in the patient.

In a particular embodiment, a patient is not-responding when his/her cognitive abilities are not improved or stabilized by said inhibitor, or even (continue to) decline despite treatment with such inhibitor. Cognitive abilities designate for example orientation, memory, executive function, registration, attention, calculation, recall, visuospatial ability, language or praxis, judgment and problem solving. The person having skills in the art is familiar with methods for assessing the patient cognitive abilities of patient. In that respect, cognitive tests have also been developed that are applicable to AD or AD related disorders. For example, the ADAS-Cog (26), MMSE (27), CDR (28), CDR-SOB or CDR-SB (29), CIBIC (30.), IDDD (31), IADL (32), ISAAC (33) or ADCOMS (34) are all tests used commonly to assess an AD or AD related disorders patient's cognitive performances. ADAS-Cog (Cognitive subscale of the Alzheimer's Disease Assessment Scale) is a test assessing orientation, memory, executive function, visuospatial ability, language or praxis, with a range of scores from 0 to 70, a higher score indicates great impairment. According to this test, an increase of the score between two consecutive tests reflects an increased impairment of the patient's cognitive functions. In a particular embodiment, a patient is not-responding (or has lost responsiveness) to an inhibitor if his score in ADAS-cog method increases by at least 5% between two tests carried out at 1 month time interval. The MMSE (Mini-Mental State Examination) is a test assessing orientation, registration, attention, and calculation, recall, and language. It has a range of scores from 0 to 30, a higher score indicated better cognitive functions. According to this test a decrease of the score between two consecutive tests reflects an increased impairment of the patient's cognitive functions. In a particular embodiment, a patient is not-responding (or has lost responsiveness) to an inhibitor if his score in MMSE method decreases by at least 5% between two tests carried out at 1 month time interval. CDR-SOB/CDR-SB (Clinical Dementia Rating Scale—Sum of Box) is a test assessing the patient's ability to function in six cognitive categories being memory, orientation, judgment and problem solving, community affairs/ involvement, home life and hobbies, and personal care, with a range of scores from 0 to 18, a higher score indicates greater impairment. According to this test, an increase of the score between two consecutive tests reflects an increased impairment of the patient's cognitive functions. In a particular embodiment, a patient is not-responding (or has lost responsiveness) to an inhibitor if his score in CDR-SOB method increases by at least 5% between two tests carried out at 1 month time interval. In order to assess variation of cognitive performances in a patient, at least two consecutive tests should be performed. The period between two tests can be 1, 2, 3 or 4 weeks, 1, 2, 3, 4, 5, 6, 7, 8, 9, 10, 11 or 12 months, or 1 or 2 years. A patient having AD or AD related disorders may also be regarded to be non-responding to an acetylcholinesterase inhibitor when said patient has been treated with an inhibitor of acetylcholinesterase for a period of at least 12 weeks with no improvement or stabilization of a cognitive function, preferably at least 4, 5 or 6 months, even more preferably for at least 1, 2 or 3 years. As evidence in clinical trials conducted in Alzheimer's patient with an acetylcholinesterase inhibitor, use of said inhibitor provides an improvement of cognitive function for the first 12 weeks, then the patient's cognitive function starts declining again, and the level of cognitive impairment of the patient before treatment is usually reached only 6 months after initiation of the treatment. This has been demonstrated for example with donepezil (20-23), rivastigmine (24) and galantamine (25).

Within the context of this invention, the designation of a specific drug or compound is meant to include not only the specifically named molecule, but also any pharmaceutically acceptable salt, hydrate, derivative, isomer, racemate, conjugate, prodrug or derivative thereof of any chemical purity.

The term “combination or combinatorial treating/therapy” designates a treatment wherein at least Baclofen and Acamprosate are co-administered to a subject to cause a biological effect. Likewise, the “combination or combinatorial treating/therapy” designates a treatment wherein at least Baclofen, Acamprosate and acetylcholinesterase inhibitor, more particular donepezil, rivastigmine or galantamine are co-administered to a subject to cause a biological effect. In a combined therapy according to this invention, the at least two or three drugs may be administered together or separately, at the same time or sequentially. Also, the at least two or three drugs may be administered through different routes and protocols. As a result, although they may be formulated together, the drugs of a combination may also be formulated separately.

The term “prodrug” as used herein refers to any functional derivatives (or precursors) of a compound of the present invention, which, when administered to a biological system, generates said compound as a result of e.g., spontaneous chemical reaction(s), enzyme catalysed chemical reaction(s), and/or metabolic chemical reaction(s). Prodrugs are usually inactive or less active than the resulting drug and can be used, for example, to improve the physicochemical properties of the drug, to target the drug to a specific tissue, to improve the pharmacokinetic and pharmacodynamic properties of the drug and/or to reduce undesirable side effects. Some of the common functional groups that are amenable to prodrug design include, but are not limited to, carboxylic, hydroxyl, amine, phosphate/phosphonate and carbonyl groups. Prodrugs typically produced via the modification of these groups include, but are not limited to, esters, carbonates, carbamates, amides and phosphates. Specific technical guidance for the selection of suitable prodrugs is general common knowledge (35-39). Furthermore, the preparation of prodrugs may be performed by conventional methods known by those skilled in the art. Methods which can be used to synthesize other prodrugs are described in numerous reviews on the subject (35-42). For example, Arbaclofen Placarbil is listed in ChemID plus Advance database (website: chem.sis.nlm.nih.gov/chemidplus/) and Arbaclofen Placarbil is a well-known prodrug of Baclofen (43-44).

The term “derivative” of a compound includes any molecule that is functionally and/or structurally related to said compound, such as an acid, amide, ester, ether, acetylated variant, hydroxylated variant, or an alkylated (C1-C6) variant of such a compound. The term derivative also includes structurally related compound having lost one or more substituent as listed above. For example, Homotaurine is a deacetylated derivative of Acamprosate. Preferred derivatives of a compound are molecules having a substantial degree of similarity to said compound, as determined by known methods. Similar compounds along with their index of similarity to a parent molecule can be found in numerous databases such as PubChem (http://pubchem.ncbi.nlm.nih.gov/search/) or DrugBank (see Worldwide Web site: drugbank.ca/)(45). In a more preferred embodiment, derivatives should have a Tanimoto similarity index greater than 0.4, preferably greater than 0.5, more preferably greater than 0.6, even more preferably greater than 0.7 with a parent drug. The Tanimoto similarity index is widely used to measure the degree of structural similarity between two molecules. Tanimoto similarity index can be computed by software such as the Small Molecule Subgraph Detector (46-47) available online (see Worldwide Web site: ebi.ac.uk/thornton-srv/software/SMSD/). Preferred derivatives should be both structurally and functionally related to a parent compound, i.e., they should also retain at least part of the activity of the parent drug, more preferably they should have a protective activity against Aβ.

The term derivatives also include metabolites of a drug, e.g., a molecule which results from the (biochemical) modification(s) or processing of said drug after administration to an organism, usually through specialized enzymatic systems, and which displays or retains a biological activity of the drug. Metabolites have been disclosed as being responsible for much of the therapeutic action of the parent drug. In a specific embodiment, a “metabolite” as used herein designates a modified or processed drug that retains at least part of the activity of the parent drug, preferably that has a protective activity against Aβ toxicity.

The term “salt” refers to a pharmaceutically acceptable and relatively non-toxic, inorganic or organic acid addition salt of a compound of the present invention. Pharmaceutical salt formation consists in pairing an acidic, basic or zwitterionic drug molecule with a counterion to create a salt version of the drug. A wide variety of chemical species can be used in neutralization reaction. Pharmaceutically acceptable salts of the invention thus include those obtained by reacting the main compound, functioning as a base, with an inorganic or organic acid to form a salt, for example, salts of acetic acid, nitric acid, tartric acid, hydrochloric acid, sulfuric acid, phosphoric acid, methane sulfonic acid, camphor sulfonic acid, oxalic acid, maleic acid, succinic acid or citric acid. Pharmaceutically acceptable salts of the invention also include those in which the main compound functions as an acid and is reacted with an appropriate base to form, e.g., sodium, potassium, calcium, magnesium, ammonium, or choline salts. Though most of salts of a given active principle are bioequivalents, some may have, among others, increased solubility or bioavailability properties. Salt selection is now a common standard operation in the process of drug development as taught by H. Stahl and C. G. Wermuth in their handbook (48).

In a preferred embodiment, the designation of a compound is meant to designate the compound per se, as well as any pharmaceutically acceptable salt, hydrate, isomer, racemate, ester or ether thereof.

In a more preferred embodiment, the designation of a compound is meant to designate the compound as specifically designated per se, as well as any pharmaceutically acceptable salt thereof.

In a particular embodiment, a sustained-release formulation of the compound is used.

As discussed above, the invention relates to particular drug combinations which have a strong unexpected protective effect against Aβ toxicity and/or improvement of cognitive performances involved in AD or AD related disorders in subject treated with an acetylcholinesterase inhibitor and having lost responsiveness to said acetylcholinesterase inhibitor. These drug combinations therefore represent novel approaches for treating AD or AD related disorders in subject treated with acetylcholinesterase inhibitor having lost responsiveness to said acetylcholinesterase inhibitor. More specifically, the invention discloses compositions, comprising Baclofen in combination with Acamprosate, which provide a significant effect in vivo on AD or AD related disorders in subject treated with acetylcholinesterase inhibitor having lost responsiveness to said acetylcholinesterase inhibitor.

Indeed, the invention shows, in the experimental part, that combination therapies comprising Baclofen and Acamprosate can substantially improve the condition of patients afflicted with AD or AD related disorders, wherein said patients are already treated with acetylcholinesterase inhibitor and have lost responsiveness to said acetylcholinesterase inhibitor. In particular, the inventors have surprisingly discovered that Baclofen and Acamprosate combinations have a strong, unexpected effect on cognitive performance in ICV beta-amyloid intoxicated animals treated with acetylcholinesterase inhibitor having lost responsiveness to said acetylcholinesterase inhibitor, and represent new therapeutic approaches of AD or AD related disorders in subject treated with acetylcholinesterase inhibitor having lost responsiveness to said acetylcholinesterase inhibitor. As disclosed in the examples, composition therapies using at least Baclofen and Acamprosate have a strong unexpected effect on cognitive functions in an animal intoxicated with Abeta peptide and already treated with an acetylcholinesterase inhibitor, more particularly donepezil at a therapeutic effective dose. More importantly, these combinations showed in vivo that the combination of baclofen and Acamprosate resulted in immediate rescue of cognitive functions at a stage where the animals became irresponsive to the acetylcholinesterase inhibitor. This combination therefore represents novel approaches for treating AD or AD related disorders in subject treated with an acetylcholinesterase inhibitor and that has lost responsiveness to said acetylcholinesterase inhibitor.

The present invention therefore proposes a novel therapy for AD or AD related disorders in subject treated with acetylcholinesterase inhibitor and that have lost responsiveness to said acetylcholinesterase inhibitor, based on Baclofen and Acamprosate compositions.

In a further embodiment, the invention relates to a composition comprising Baclofen and Acamprosate for use in the treatment of AD or AD related disorders in subject treated with acetylcholinesterase inhibitor having lost responsiveness to said acetylcholinesterase inhibitor.

In a further embodiment, the invention relates to the use of Baclofen and Acamprosate for the manufacture of a medicament for the treatment of AD or AD related disorders in subject treated with acetylcholinesterase inhibitor having lost responsiveness to said acetylcholinesterase inhibitor.

The present invention also proposes a novel therapy for AD or AD related disorders in subject treated with acetylcholinesterase inhibitor and that have lost responsiveness to said acetylcholinesterase inhibitor, based on Baclofen and Acamprosate compositions and wherein said subject is regarded to be non-responding to said inhibitor of acetylcholinesterase if the performance of said subject in a cognitive test result in an increase impairment of the patient cognitive functions in comparison with a previous performance of said subject in a same cognitive test.

In a further embodiment, the invention relates to a composition comprising Baclofen and Acamprosate for use in the treatment of AD or AD related disorders in subject treated with acetylcholinesterase inhibitor having lost responsiveness to said acetylcholinesterase inhibitor, and wherein said subject is regarded to be non-responding to said inhibitor of acetylcholinesterase if the performance of said subject in a cognitive test result in an increase impairment of the patient cognitive functions in comparison with a previous performance of said subject in a same cognitive test.

In a further embodiment, the invention relates to the use of Baclofen and Acamprosate for the manufacture of a medicament for the treatment of AD or AD related disorders in subject treated with acetylcholinesterase inhibitor having lost responsiveness to said acetylcholinesterase inhibitor, and wherein said subject is regarded to be non-responding to said inhibitor of acetylcholinesterase if the performance of said subject in a cognitive test result in an increase impairment of the patient cognitive functions in comparison with a previous performance of said subject in a same cognitive test.

The present invention further proposes a novel therapy for AD or AD related disorders in subject treated with acetylcholinesterase inhibitor and that have lost responsiveness to said acetylcholinesterase inhibitor, based on Baclofen and Acamprosate compositions, wherein the subject is deemed to have lost responsiveness to the treatment with said acetylcholinesterase inhibitor after a period of at least 12 weeks.

In a further embodiment, the invention relates to a composition comprising Baclofen and Acamprosate for use in the treatment of AD or AD related disorders in subject treated with acetylcholinesterase inhibitor having lost responsiveness to said acetylcholinesterase inhibitor, wherein the subject is deemed to have lost responsiveness to the treatment with said acetylcholinesterase inhibitor after a period of at least 12 weeks.

In a further embodiment, the invention relates to the use of Baclofen and Acamprosate for the manufacture of a medicament for the treatment of AD or AD related disorders in subject treated with acetylcholinesterase inhibitor having lost responsiveness to said acetylcholinesterase inhibitor, wherein the subject is deemed to have lost responsiveness to the treatment with said acetylcholinesterase inhibitor after a period of at least 12 weeks.

Illustrative CAS numbers for Baclofen and Acamprosate are provided in Table 1 below. Table 1 cites also, in a non-limitative way, common salts, racemates, prodrugs, metabolites or derivatives for these compounds used in the compositions of the invention.

TABLE 1

| | | |
|---|---|---|
| | | Class or |
| | | Tanimoto |
| Drug | CAS Numbers | similarity index |

Acamprosate and related compounds

| | | |
|---|---|---|
| Acamprosate | 77337-76-9; | NA |
| | 77337-73-6 | |
| Homotaurine | 3687-18-1 | 0.73 |
| Ethyl Dimethyl Ammonio | / | 0.77 |
| Propane Sulfonate | | |
| Taurine | 107-35-7 | 0.5 |

Baclofen and related compounds

| | | |
|---|---|---|
| Baclofen | 1134-47-0; 66514-99-6; | NA |
| | 69308-37-8; 70206-22-3; | |
| | 63701-56-4; 63701-55-3 | |
| 3-(p-chlorophenyl)-4- | / | Metabolite |
| hydroxybutyric acid | | |
| Arbaclofen placarbil | 847353-30-4 | Prodrug |

Specific examples of prodrugs of Baclofen are given in Hanafi et al., 2011 (49), particularly Baclofen esters and Baclofen ester carbamates, which are of particular interest for CNS targeting. Hence such prodrugs are particularly suitable for compositions of this invention. Baclofen placarbil as mentioned before is also a well-known prodrug and may thus be used instead of Baclofen in compositions of the invention. Other prodrugs of Baclofen can be found in the following patent applications: WO2010102071, US2009197958, WO2009096985, WO2009061934, WO2008086492, US2009216037, WO2005066122, US2011021571, WO2003077902, WO2010120370.

Useful prodrugs for acamprosate such as pantoic acid ester, neopentyl sulfonyl esters, neopentyl sulfonyl esters prodrugs or masked carboxylate neopentyl sulfonyl ester prodrugs of acamprosate are notably listed in WO2009033069, WO2009033061, WO2009033054 WO2009052191, WO2009033079, US 2009/0099253, US 2009/0069419, US 2009/0082464, US 2009/0082440, and US 2009/0076147.

As emphasized, the combination of baclofen and Acamprosate may further comprises an acetylcholinesterase inhibitor, such as for example donepezil (CAS: 120014-06-4), galantamine (CAS: 357-70-0) or rivastigimine (CAS: 123441-03-2).

Accordingly, the combination of the present invention also comprises a combination of baclofen, Acamprosate and an acetylcholinesterase inhibitor, more particularly an acetylcholinesterase inhibitor selected from the list consisting of donepezil, galantamine and rivastigimine.

In a preferred embodiment, the drugs of the invention are used in combination(s) for combined, separate or sequential administration, in order to provide the most effective effect.

Preferred compositions of the invention, for use in the treatment AD or AD related disorders in subject treated with an acetylcholinesterase inhibitor and that has lost responsiveness to said acetylcholinesterase inhibitor, comprise one of the following drug combinations, for combined, separate or sequential administration:
Baclofen and Acamprosate,
Baclofen and Acamprosate and Donepezil,
Baclofen and Acamprosate and rivastigmine,
Baclofen and Acamprosate and galantamine.

In a preferred embodiment, the drugs of the invention are used in combination(s) for combined, separate or sequential administration, in order to provide the most effective effect.

In another preferred embodiment, the subject in need thereof is a subject having AD or AD related disorders, wherein said subject is already treated with a therapeutic dose of an inhibitor of acetylcholinesterase and said subject is not responding anymore to said inhibitor of acetylcholinesterase, more particularly said acetylcholinesterase inhibitor is selected from the list consisting of donepezil, rivastigmine and galantamine, even more preferably donepezil.

In another preferred embodiment, the subject in need thereof is a subject having AD or AD related disorders, wherein said subject is already treated with a therapeutic dose of an inhibitor of acetylcholinesterase and said subject is not responding anymore to said inhibitor of acetylcholinesterase, wherein said subject is regarded to be non-responding to said inhibitor of acetylcholinesterase if the performance of said subject in a cognitive test result in an increase impairment of the patient cognitive functions in comparison with a previous performance of said subject in a same cognitive test.

Specific examples of cognitive functions assessed by the cognitive tests for use in the invention are orientation, memory, executive function, registration, attention, calculation, recall, visuospatial ability, language and praxis.

Specific examples of cognitive tests for use in the invention are the ADAS-Cog, MMSE, CDR, CDR-SOB/SB, CIBIC, IDDD, QoL, IADL, ISAAC or ADCOMS.

The period between two consecutive tests can be 1, 2, 3 or 4 weeks, 1, 2, 3, 4, 5, 6, 7, 8, 9, 10, 11 or 12 months, or 1 or 2 years.

In a further preferred embodiment, the subject in need thereof is a subject having AD or AD related disorders, wherein said subject is already treated with a therapeutic dose of an inhibitor of acetylcholinesterase and said subject is not responding anymore to said inhibitor of acetylcholinesterase, wherein said subject is regarded to be non-responding to said inhibitor of acetylcholinesterase if the said subject has been treated with an inhibitor of acetylcholinesterase for a period of at least 12 weeks, preferably 4, 5 to 6 months, even more preferably for at least 1, 2 or 3 years.

An object of this invention thus also resides in a composition as defined above for treating AD or AD related disorders in human subjects as defined above.

As indicated previously, in a combination therapy of this invention, the compounds or drugs may be formulated together or separately, and administered together, separately or sequentially.

A further object of this invention resides in the use of a composition as defined above for the manufacture of a medicament for treating AD or AD related disorders in human subjects as defined above.

The invention further provides a method for treating AD or AD related disorders in human subjects as defined above, comprising administering to a subject in need thereof an effective amount of a composition as disclosed above.

A further object of the invention is a method of treating AD or AD related disorders in human subjects as defined above, the method comprising simultaneously, separately or sequentially administering to a subject in need thereof an effective amount of a composition as disclosed above.

In a preferred embodiment, the invention relates to a method of treating AD or AD related disorders in human subjects as defined above in a subject in need thereof, comprising administering simultaneously, separately or sequentially to the subject an effective amount of Baclofen and Acamprosate.

The compositions of the invention typically comprise one or several pharmaceutically acceptable carriers or excipients. Also, for use in the present invention, the drugs or compounds are usually mixed with pharmaceutically acceptable excipients or carriers.

In this regard, a further object of this invention is a method of preparing a pharmaceutical composition, the method comprising mixing the above compounds in an appropriate excipient or carrier.

In a particular embodiment, the method comprises mixing Baclofen and Acamprosate in an appropriate excipient or carrier.

According to preferred embodiments of the invention, as indicated above, the compounds are used as such or in the form of a pharmaceutically acceptable salt, prodrug, derivative, or sustained release formulation thereof.

Although very effective in vivo, depending on the subject or specific condition, the combination therapy of the invention may further be used in conjunction or association or combination with additional drugs or treatments beneficial to the treated condition in the subjects.

Therapy according to the invention may be provided at home, the doctor's office, a clinic, a hospital's outpatient department, or a hospital, so that the doctor can observe the therapy's effects closely and make any adjustments that are needed.

The duration of the therapy depends on the stage of the disease being treated, age and condition of the patient, and how the patient responds to the treatment. The dosage, frequency and mode of administration of each component of the combination can be controlled independently. For example, one drug may be administered orally while the second drug may be administered intramuscularly. Combination therapy may be given in on-and-off cycles that include rest periods so that the patient's body has a chance to recovery from any as yet unforeseen side-effects. The drugs may also be formulated together such that one administration delivers all drugs.

The administration of each drug of the combination may be by any suitable means that results in a concentration of the drug that, combined with the other component, is able to ameliorate the patient condition or efficiently treat the disease or disorder.

While it is possible for the drugs the combination to be administered as the pure chemical it is preferable to present them as a pharmaceutical composition, also referred to in this context as pharmaceutical formulation. Possible compositions include those suitable for oral, rectal, topical (including transdermal, buccal and sublingual), or parenteral (including subcutaneous, intramuscular, intravenous and intradermal) administration.

More commonly these pharmaceutical formulations are prescribed to the patient in “patient packs” containing a number dosing units or other means for administration of metered unit doses for use during a distinct treatment period in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions. Thus, the invention further includes a pharmaceutical formulation, as herein before described, in combination with packaging material suitable for said formulations. In such a patient pack the intended use of a formulation for the combination treatment can be inferred by instructions, facilities, provisions, adaptations and/or other means to help using the formulation most suitably for the treatment. Such measures make a patient pack specifically suitable for and adapted for use for treatment with the combination of the present invention.

The drug may be contained, in any appropriate amount, in any suitable carrier substance. The drug may be present in an amount of up to 99% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols.

The pharmaceutical compositions may be formulated according to conventional pharmaceutical practice (see, e.g. 50, 51).

Pharmaceutical compositions according to the invention may be formulated to release the active drug substantially immediately upon administration or at any predetermined time or time period after administration.

The controlled release formulations include (i) formulations that create a substantially constant concentration of the drug within the body over an extended period of time; (ii) formulations that after a predetermined lag time create a substantially constant concentration of the drug within the body over an extended period of time; (iii) formulations that sustain drug action during a predetermined time period by maintaining a relatively, constant, effective drug level in the body with concomitant minimization of undesirable side effects associated with fluctuations in the plasma level of the active drug substance; (iv) formulations that localize drug action by, e.g., spatial placement of a controlled release composition adjacent to or in the diseased tissue or organ; and (v) formulations that target drug action by using carriers or chemical derivatives to deliver the drug to a particular target cell type.

Administration of drugs in the form of a controlled release formulation is especially preferred in cases in which the drug has (i) a narrow therapeutic index (i.e., the difference between the plasma concentration leading to harmful side effects or toxic reactions and the plasma concentration leading to a therapeutic effect is small; in general, the therapeutic index, TI, is defined as the ratio of median lethal dose (LD50) to median effective dose (ED50)); (ii) a narrow absorption window in the gastro-intestinal tract; or (iii) a very short biological half-life so that frequent dosing during a day is required in order to sustain the plasma level at a therapeutic level.

Any of a number of strategies can be pursued in order to obtain controlled release in which the rate of release outweighs the rate of metabolism of the drug in question. Controlled release may be obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner (single or multiple unit tablet or capsule compositions, oil solutions, suspensions, emulsions, microcapsules, microspheres, nanoparticles, patches, and liposomes).

### Solid Dosage Forms for Oral Use

Formulations for oral use include tablets containing the composition of the invention in a mixture with non-toxic pharmaceutically acceptable excipients. These excipients may be, for example, inert diluents or fillers (e.g., sucrose, microcrystalline cellulose, starches including potato starch, calcium carbonate, sodium chloride, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., acacia, alginic acid, sodium alginate, gelatin, starch, pregelatinized starch, microcrystalline cellulose, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., stearic acid, silicas, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug substance in a predetermined pattern (e.g., in order to achieve a controlled release formulation) or it may be adapted not to release the active drug substance until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). A time delay material such as, e.g., glyceryl monostearate or glyceryl distearate may be employed.

The solid tablet compositions may include a coating adapted to protect the composition from unwanted chemical changes, (e.g., chemical degradation prior to the release of the active drug substance). The coating may be applied on the solid dosage form in a similar manner as that described in Encyclopedia of Pharmaceutical Technology.

Drugs may be mixed together in the tablet, or may be partitioned. For example, a first drug is contained on the inside of the tablet, and a second drug is on the outside, such that a substantial portion of the second drug is released prior to the release of the first drug.

Formulations for oral use may also be presented as chewable tablets, or as hard gelatin capsules wherein the active ingredient is mixed with an inert solid diluent (e.g., potato starch, microcrystalline cellulose, calcium carbonate, calcium phosphate or kaolin), or as soft gelatin capsules wherein the active ingredient is mixed with water or an oil medium, for example, liquid paraffin, or olive oil. Powders and granulates may be prepared using the ingredients mentioned above under tablets and capsules in a conventional manner.

Controlled release compositions for oral use may, e.g., be constructed to release the active drug by controlling the dissolution and/or the diffusion of the active drug substance.

Dissolution or diffusion controlled release can be achieved by appropriate coating of a tablet, capsule, pellet, or granulate formulation of drugs, or by incorporating the drug into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, d1-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the drugs of the claimed combinations may also be in the form of a buoyant tablet or capsule (i.e., a tablet or capsule that, upon oral administration, floats on top of the gastric content for a certain period of time). A buoyant tablet formulation of the drug(s) can be prepared by granulating a mixture of the drug(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Liquids for Oral Administration

Powders, dispersible powders, or granules suitable for preparation of an aqueous suspension by addition of water are convenient dosage forms for oral administration. Formulation as a suspension provides the active ingredient in a mixture with a dispersing or wetting agent, suspending agent, and one or more preservatives. Suitable suspending agents are, for example, sodium carboxymethylcellulose, methylcellulose, sodium alginate, and the like.

### Parenteral Compositions

The pharmaceutical composition may also be administered parenterally by injection, infusion or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation or it may be presented as a dry powder to be reconstituted with water or another suitable vehicle before use. Apart from the active drug(s), the composition may include suitable parenterally acceptable carriers and/or excipients. The active drug(s) may be incorporated into microspheres, microcapsules, nanoparticles, liposomes, or the like for controlled release. The composition may include suspending, solubilizing, stabilizing, pH-adjusting agents, and/or dispersing agents.

The pharmaceutical compositions according to the invention may be in the form suitable for sterile injection. To prepare such a composition, the suitable active drug(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl or n-propyl p-hydroxybenzoate). In cases where one of the drugs is only sparingly or slightly soluble in water, a dissolution enhancing or solubilizing agent can be added, or the solvent may include 10-60% w/w of propylene glycol or the like.

Controlled release parenteral compositions may be in form of aqueous suspensions, microspheres, microcapsules, magnetic microspheres, oil solutions, oil suspensions, or emulsions. Alternatively, the active drug(s) may be incorporated in biocompatible carriers, liposomes, nanoparticles, implants, or infusion devices. Materials for use in the preparation of microspheres and/or microcapsules are, e.g., biodegradable/bioerodible polymers such as polygalactin, poly-(isobutyl cyanoacrylate), poly(2-hydroxyethyl-L-glutamnine). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(glycolic acid) or poly(ortho esters)).

### Alternative Routes

Although less preferred and less convenient, other administration routes, and therefore other formulations, may be contemplated. In this regard, for rectal application, suitable dosage forms for a composition include suppositories (emulsion or suspension type), and rectal gelatin capsules (solutions or suspensions). In a typical suppository formulation, the active drug(s) are combined with an appropriate pharmaceutically acceptable suppository base such as cocoa butter, esterified fatty acids, glycerinated gelatin, and various water-soluble or dispersible bases like polyethylene glycols. Various additives, enhancers, or surfactants may be incorporated.

The pharmaceutical compositions may also be administered topically on the skin for percutaneous absorption in dosage forms or formulations containing conventionally non-toxic pharmaceutical acceptable carriers and excipients including microspheres and liposomes. The formulations include creams, ointments, lotions, liniments, gels, hydrogels, solutions, suspensions, sticks, sprays, pastes, plasters, and other kinds of transdermal drug delivery systems. The pharmaceutically acceptable carriers or excipients may include emulsifying agents, antioxidants, buffering agents, preservatives, humectants, penetration enhancers, chelating agents, gel-forming agents, ointment bases, perfumes, and skin protective agents.

The preservatives, humectants, penetration enhancers may be parabens, such as methyl or propyl p-hydroxybenzoate, and benzalkonium chloride, glycerin, propylene glycol, urea, etc.

The pharmaceutical compositions described above for topical administration on the skin may also be used in connection with topical administration onto or close to the part of the body that is to be treated. The compositions may be adapted for direct application or for application by means of special drug delivery devices such as dressings or alternatively plasters, pads, sponges, strips, or other forms of suitable flexible material.

### Dosages and Duration of the Treatment

It will be appreciated that the drugs of the combination may be administered concomitantly, either in the same or different pharmaceutical formulation or sequentially. If there is sequential administration, the delay in administering the second (or additional) active ingredient should not be such as to lose the benefit of the efficacious effect of the combination of the active ingredients. A minimum requirement for a combination according to this description is that the combination should be intended for combined use with the benefit of the efficacious effect of the combination of the active ingredients. The intended use of a combination can be inferred by facilities, provisions, adaptations and/or other means to help using the combination according to the invention.

Therapeutically effective amounts of the drugs in a combination of this invention include, e.g., amounts that are effective for reducing Alzheimer's disease or Alzheimer's disease related disorders symptoms, halting or slowing the progression of the disease once it has become clinically manifest.

Each of the active drugs of the present invention may be administered in single or divided doses, for example two, three or four times daily, administered together, separately or sequentially. A single daily dose of each drug in the combination is preferred, with a single daily dose of all drugs in a single pharmaceutical composition (unit dosage form) being most preferred.

Administration can be one to several times daily for several days to several years, and may even be for the life of the patient. Chronic or at least periodically repeated long-term administration is indicated in most cases.

The term “unit dosage form” refers to physically discrete units (such as capsules, tablets, or loaded syringe cylinders) suitable as unitary dosages for human subjects, each unit containing a predetermined quantity of active material or materials calculated to produce the desired therapeutic effect, in association with the required pharmaceutical carrier.

The amount of each drug in a preferred unit dosage composition depends upon several factors including the administration method, the body weight and the age of the patient, the stage of the disease, the risk of potential side effects considering the general health status of the person to be treated. Additionally, pharmacogenomic (the effect of genotype on the pharmacokinetic, pharmacodynamic or efficacy profile of a therapeutic) information about a particular patient may affect the dosage used.

Except when responding to especially impairing cases, where higher dosages may be required, the preferred dosage of each drug in the combination will usually lie within the range of doses not above the dosage usually prescribed for long-term maintenance treatment or proven to be safe in phase 3 clinical studies.

Specific examples of dosages of drugs for use in the invention are provided below:
Acamprosate between 1 and 1000 mg/day, preferably less than 400 mg per day, more preferably less than 200 mg/day, even more preferably less than 50 mg/day, such dosages being particularly suitable for oral administration.
Baclofen between 0.01 to 150 mg per day, preferably less than 100 mg per day, more preferably less than 50 mg/day, even more preferably less than 25 mg/day, such dosages being particularly suitable for oral administration.
Donepezil between 0.1 and 100 mg/day, preferably between 0.5 and 50 mg/day, more preferably between 1 and 20 mg/day, more preferably between 4 and 15 mg/day, and even more preferably 5 mg/day or 10 mg/day, such dosages being particularly suitable for oral administration.
Galantamine between 0.1 and 100 mg/day, preferably between 1 and 50 mg/day, more preferably between 8 and 40 mg/day, more preferably between 16 and 32 mg/day, and even more preferably 24 mg/day, such dosages being particularly suitable for oral administration.
Rivastigmine between 0.1 to 100 mg/day, preferably between 0.5 to 50 mg/day, more preferably between 1 to 30 mg/days, more preferably between 3 to 18 mg/day, even more preferably 3 mg/day, 6 mg/day, 9 mg/day or 18 mg/day.

In the composition of the present invention, baclofen and acamprosate may be used in different ratios, e.g., at a weight ratio Acamprosate/Baclofen comprised between from 0.05 to 1000 (W:W), preferably between 0.05 to 100 (W:W), more preferably between 0.05 to 50 (W:W).

It will be understood that the amount of the drug actually administered will be determined by a physician, in the light of the relevant circumstances including the condition or conditions to be treated, the exact composition to be administered, the age, weight, and response of the individual patient, the severity of the patient's symptoms, and the chosen route of administration. Therefore, the above dosage ranges are intended to provide general guidance and support for the teachings herein, but are not intended to limit the scope of the invention.

The following examples are given for purposes of illustration and not by way of limitation.

### EXAMPLES

### A) Baclofen and Acamprosate Treatment of Diseases Related to Aβ Toxicity in In Vivo Model Non-Responsive to Therapeutic Effective Dose of Donepezil

### Protocol

### Animals

Male Swiss mice weighing 30-35 g were purchase from JANVIER (Saint Berthevin, France). Housing and experiments were performed within AMYLGEN's animal facility (Direction Régionale de l′Alimentation, de l'Agriculture et de la Forêt du Languedoc-Roussillon, agreement #A 34-169-002 from May 2, 2014). Animals were housed in groups with access to food and water ad libitum, except during behavioral experiments. The temperature and humidity were controlled, and the animal facility on a 12 h/12 h light/dark cycle (lights off at 07:00 pm). Mice were numbered by marking their tail using permanent markers. All animal procedures will be conducted in strict adherence to the European Union Directive of Sep. 22, 2010 (2010/63/UE).

Health of animals, general aspect of animals and activity were checked daily. Weight was monitored 3 times per week. Acute or delayed mortality were checked.

### Amyloid Peptide Injection (By ICV)

Male Swiss mice were anesthetized 5 minutes with isoflurane 2.5%. At Day 01, animals were injected intracerebroventricularly (ICV) through a 28-gauge stainless-steel needle, 4 mm long. An injection volume of 3 μl was delivered gradually within 30 s and the needle left in place for an additional 30 s before being removed. Animals were injected with amyloid peptide 25-35 (Aβ₂₅₋₃₅) peptide (9 nmol/mouse) or Scrambled Aβ peptide (Sc.Aβ) (9 nmol/mouse), in a final volume of 3 μl mouse, according to the previously described method (52-56). Homogeneous preparation of the Aβ₂₅₋₃₅ peptide were performed according to the AMYLGEN's own procedure.

### Treatment

All animals received a per os gavage using an inox steel cannula (5 mL/Kg). All the treatments were administered under a volume calculated according to the individual body weight of each mouse (5 mL/Kg). Vehicle and donepezil (1 mg/Kg) administration were done once a day, and the mix Acamprosate/baclofen twice a day (0.2 mg/Kg and 3 mg/Kg, respectively).

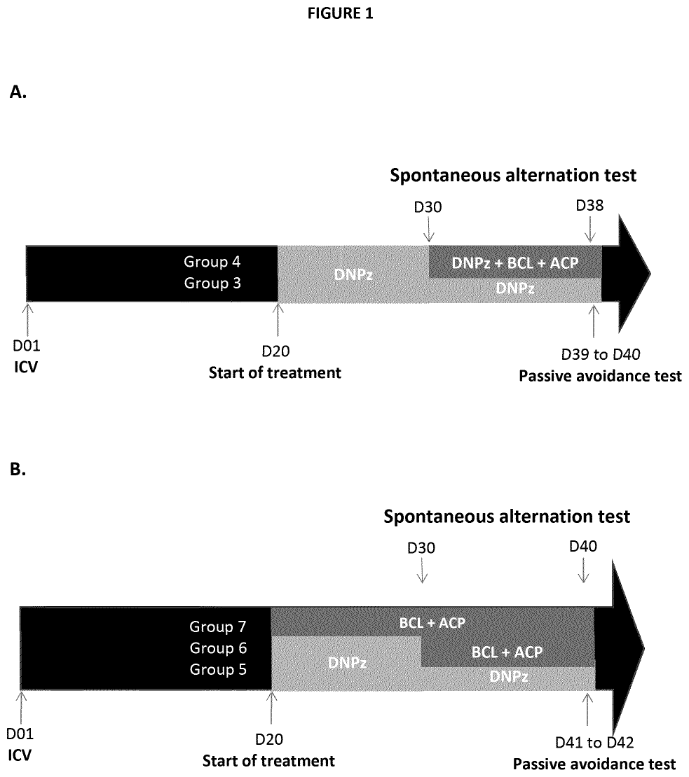

In a first set of experiments (FIG. 1A), the treatment, donepezil (1 mg/Kg), was started 19 days (D20) after icy injection for 21 days (D40) (group 3 and 4). At D30 one group of animals treated with donepezil was administered with acamprosate and baclofen (0.2 mg/Kg and 3 mg/Kg, respectively), in addition of donepezil treatment (group 4).

In a second set of experiments (FIG. 1B). The treatment, donepezil (1 mg/kg) (Group 5) or the combination acamprosate and baclofen (0.2 mg/Kg and 3 mg/Kg, respectively) (group 7), was started 19 days (D20) after icy injection and maintained for 23 days (D42) in two groups of animals. In a third group of animals (group 6), initially treated with donepezil, treatment with donepezil was stopped at D30 and replaced with administration of acamprosate and baclofen (0.2 mg/Kg and 3 mg/Kg, respectively) until D42.

### Spontaneous Alternation Performance (Y-maze)

Mice were tested for spontaneous alternation performance in the Y-maze, an index of spatial working memory. The Y-maze was designed according to Itoh et al.,1993 (57) and Hiramatsu and Inoue, 1999 (58), and is made of grey polyvinylchloride. Each arm is 40 cm long, 13 cm high, 3 cm wide at the bottom, 10 cm wide at the top, and converging at an equal angle. Each mouse was placed at the end of one arm and allowed to move freely through the maze during an 8 min session. The series of arm entries, including possible returns into the same arm, were checked visually by an experimenter blind to treatment. An alternation is defined as entries into all three arms on consecutive occasions. The number of maximum alternations were therefore the total number of arm entries minus two and the percentage of alternation as calculated as (actual alternations / maximum alternations)×100. Calculated parameters consisted in the percentage of alternation (memory index) and the total number of arm entries (exploration index) (52-56).

Mice that showed an extreme behavior (Alternation percentage<20% or >90% or number of arm entries<8) were discarded. In the first set of experiments (FIG. 1A), animals were tested after the beginning of the treatment at D30 and D38. In the second set of experiments (FIG. 1B), animals were tested after the beginning of the treatment, at D30 and D40.

### Passive Avoidance Test (STPA)

All animals were tested for passive avoidance performance, an index of contextual long-term memory. The apparatus is a two-compartment (15×20×15 cm high) box with one illuminated with white polyvinylchloride walls and the other darkened with black polyvinylchloride walls and a grid floor. A guillotine door separates each compartment. A 60 W lamp positioned 40 cm above the apparatus lights up the white compartment during the experiment. Scrambled footshocks (0.3 mA for 3 s) can be delivered to the grid floor using a shock generator scrambler (MedAssociates, USA). The guillotine door was initially closed during the training session. Each mouse was placed into the white compartment. After 5 s, the door was raised. When the mouse was entered in the darkened compartment and placed all its paws on the grid floor, the door was closed and the footshocks delivered for 3 s. The step-through latency, that is, the latency spent to enter the darkened compartment, and the number of vocalizations were recorded. The retention test was carried out 24 h after training. Each mouse was placed again into the white compartment. After 5 s, the door was raised. The step-through latency was recorded up to a cut-off time of 300 s (52-56).

In the first set of experiments (FIG. 1A), animals were tested after the beginning of the treatment at D39 and D40. In the second set of experiments (FIG. 1B), animals were tested after the beginning of the treatment, at D41 and D42.

### Statistical Analyses

All values were expressed as mean±S.E.M. Statistical analyses were performed on the different conditions using one-way ANOVA (F value), followed by the Dunnett's post-hoc multiple comparison test. Passive avoidance latencies do not follow a Gaussian distribution, since upper cut-off times are set. They were therefore be analyzed using a Kruskal-Wallis non-parametric ANOVA (H value), followed by a Dunn's multiple comparison test. p<0.05 will be considered as statistically significant.

### Results

On day 1, all animals received an ICV injection whether with Aβ or Sc.Aβ. The treatments with donepezil, with a combination of acamprosate and baclofen, or with the vehicle started 19 days after the ICV (D20) to D40, when the pathology was already manifested. At D30 one group of animals treated with donepezil were supplemented with Acamprosate and baclofen for at least 11 days (D30 to D40 or D42). At D30 another group of animals also treated with donepezil stopped treatment with donepezil and instead was administered a combination of acamprosate and baclofen. The cognitive performances of animals were tested at D30 and D38 or D40 by the spontaneous alternation performance test, and at D39-40 or D41-42 for the passive avoidance test (FIG. 1A and 1B).

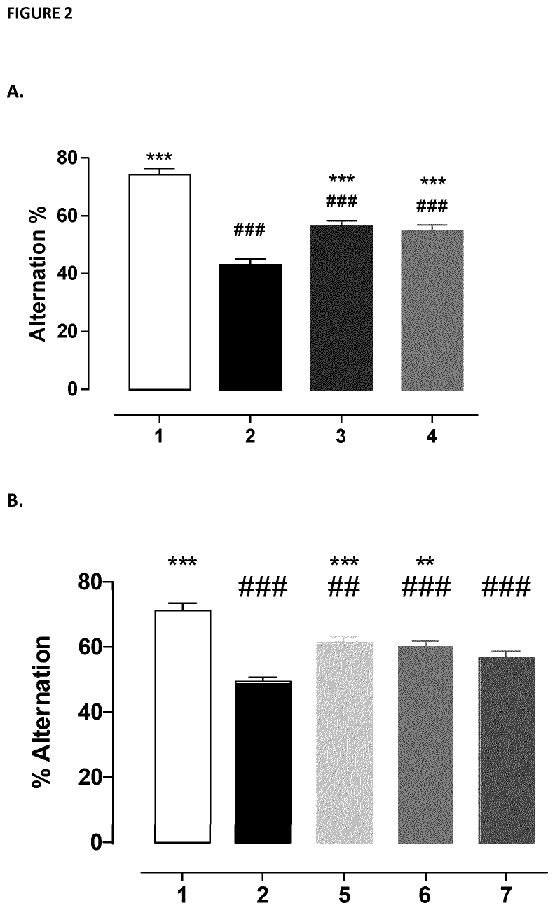

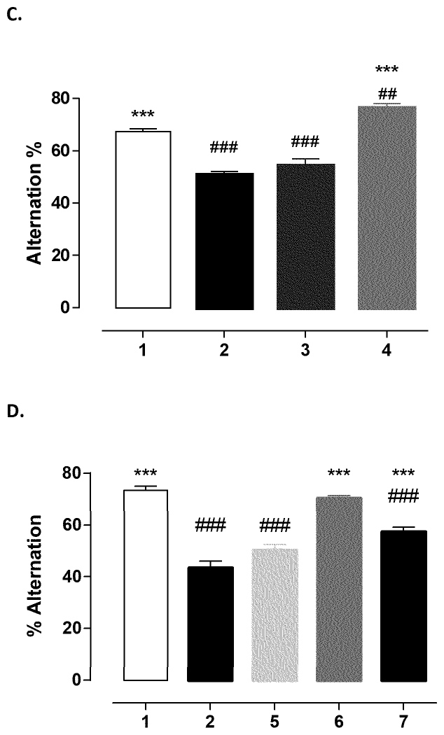

The spontaneous alternation performance assessed by Y-maze is a readout of the spatial working memory of animals. It has been shown that one ICV injection of Aβ₂₅₋₃₅ is able to induce cognitive impairment compared to Sc.Aβ ICV injection. Eleven days of treatment, started at D20 with donepezil (1 mg/Kg), or a combination of acamprosate and baclofen (0.2 mg/Kg and 3 mg/Kg, respectively), partially alleviated Aβ₂₅₋₃₅-induced cognitive impairment (FIGS. 2A-2B). The activity of donepezil was totally lost after a longer treatment period (D20 to D38 or D40) (FIGS. 2C-2D). The activity of the combination of acamprosate and baclofen (0.2 mg/Kg and 3 mg/Kg, respectively) improved after a longer treatment period (FIGS. 2B-2D). Remarkably, 10 days of donepezil treatment followed with up to 11 days period of Acamprosate and baclofen (0.2 mg/kg; 3 mg/Kg, respectively), alone (FIG. 2D) or as a supplement to donepezil (FIG. 2C), allowed to fully rescue the loss of cognitive impairment induced by Aβ₂₅₋₃₅.

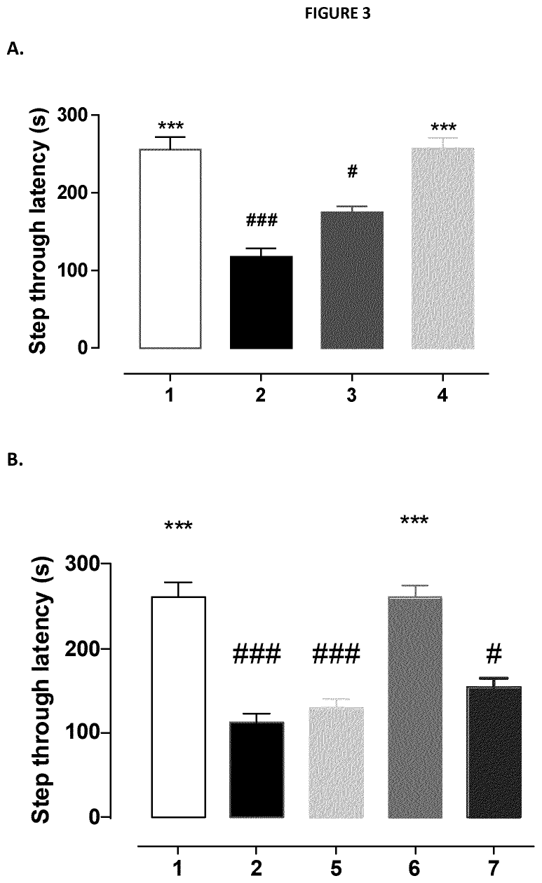

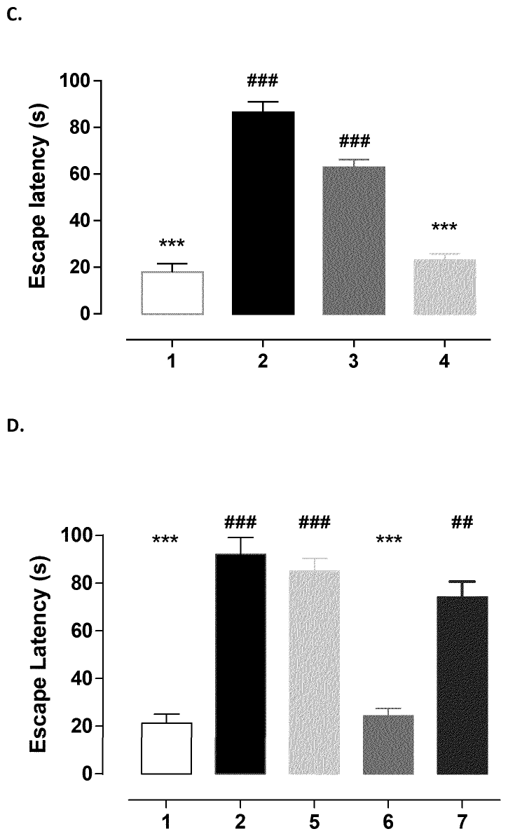

The passive avoidance test is a readout of the fear conditioning memory and implicated also in long-term memory. It has been shown that one ICV injection of Aβ₂₅₋₃₅ is able to induce cognitive impairment compared to Sc.Aβ ICV injection. The step-through latency and the escape latency of animals treated with donepezil (1 mg/Kg) or a combination of acamprosate and baclofen (0.2 mg/Kg and 3 mg/Kg, respectively) (D20 to D40 or D42) were significantly smaller than Sc.Aβ injected animal group+vehicle. This data suggested that donepezil (1 mg/Kg) alone or a combination of baclofen and acamprosate (0.2 mg/Kg and 3 mg/Kg, respectively) alone partially restored long-term memory when administered at D20 under the tested conditions (FIG. 3). Remarkably, animals initially treated with donepezil (1 mg/Kg) and administered with acamprosate and baclofen (0.2 mg/Kg and 3 mg/Kg, respectively) (D30 to D40-D42) alone (FIG. 3B-3D) or as a supplementation to donepezil treatment (FIG. 3A-3C), showed comparable performances on fear conditioning memory assessed by passive avoidance test than Sc.Aβ injected animal group+vehicle.

### Conclusion

DNPz (1 mg/Kg) administered between D20 to D30 showed a partial activity to rescue Aβ₂₅₋₃₅-induced cognitive impairment assessed by Y-maze. At D38-D40, this effect was not sustained.

The combination of acamprosate and baclofen (0.2 mg/Kg and 3 mg/Kg, respectively) administered between D20 to D30 was able to partially rescue Aβ₂₅₋₃₅-induced cognitive impairment assessed by Y-maze. This effect was substantially improved at D40.

When animals were treated with donepezil (1 mg/Kg) for 10 days (D20 to D29) and then with acamprosate and baclofen (0.2 mg/Kg and 3 mg/Kg, respectively) for 13 other days (D30 to D42), the treatment with acamprosate and baclofen was able to fully rescue Aβ₂₅₋₃₅-induced cognitive impairment assessed by Y-maze and passive avoidance test.

Likewise, when animals were treated with donepezil (1 mg/Kg) for 21 days (D20 to D40) and supplemented with acamprosate and baclofen (0.2 mg/Kg and 3 mg/Kg, respectively) for 11 subsequent days (D30 to D40), the combinational treatment was able to fully rescue Aβ₂₅₋₃₅-induced cognitive impairment assessed by Y-maze and passive avoidance test.

These data demonstrated that administration of acamprosate and baclofen, alone or as an add-on therapy to donepezil treatment, was able to fully rescue cognitive impairments in mice who lost their responsiveness to donepezil treatment.

This result was especially surprising considering that in the same model of Aβ₂₅₋₃₅-induced cognitive impairment, the sole administration of a combination of baclofen and acamprosate at the same dose and during the same period of treatment was not able to fully rescue cognitive impairments of the mice.

### B) Donepezil Treatment of Diseases Related to Aβ Toxicity in In Vivo Model

### Protocol

### Animals

Male Swiss mice weighing 30-35 g, were purchased at JANVIER (Saint Berthevin, France). Housing and experiments were performed within AMYLGEN's animal facility (Direction Régionale de l′Alimentation, de l′Agriculture et de la Forêt du Languedoc-Roussillon, agreement #A 34-169-002 from May 2, 2014). Animals were housed in groups with access to food and water ad libitum, except during behavioral experiments. The temperature and humidity were controlled, and the animal facility on a 12 h/12 h light/dark cycle (lights off at 07:00 pm). Mice were numbered by marking their tail using permanent markers. All animal procedures will be conducted in strict adherence to the European Union Directive of Sep. 22, 2010 (2010/63/UE).

Health of animals, general aspect of animals and activity were checked daily. Weight was monitored 3 times per week. Acute or delayed mortality were checked.

### Amyloid Peptide Injection (By ICV)

Male Swiss mice were anesthetized 5 minutes with isoflurane 2.5%. At day 01 (D01), animals were injected intracerebroventricularly (ICV) through a 28-gauge stainless-steel needle, 4 mm long. An injection volume of 3 μl was delivered gradually within 30 s and the needle left in place for an additional 30 s before being removed. Animals were injected with amyloid peptide 25-35 (Aβ₂₅₋₃₅) peptide (9 nmol/mouse) or Scrambled AP peptide (Sc.Aβ) (9 nmol/mouse), in a final volume of 3 μl/mouse, according to the previously described method (52-56). Homogeneous preparation of the Aβ₂₅₋₃₅ peptide were performed according to the AMYLGEN's owned procedure.

### Treatment

All animals received a per os gavage using an inox steel cannula. Vehicle or Donepezil (1 mg/kg) were administered at D8 until D17; or at D20 until D30 or at D20 to D40 once a day. All the treatments were administered under a volume calculated according to the individual body weight of each mouse (5 mL/Kg).

### Spontaneous Alternation Performance (Y-maze)

Mice were tested for spontaneous alternation performance in the Y-maze, an index of spatial working memory. The Y-maze was designed according to Itoh et al.,1993 (57) and Hiramatsu and Inoue, 1999 (58), and is made of grey polyvinylchloride. Each arm is 40 cm long, 13 cm high, 3 cm wide at the bottom, 10 cm wide at the top, and converging at an equal angle. Each mouse was placed at the end of one arm and allowed to move freely through the maze during an 8min session. The series of arm entries, including possible returns into the same arm, were checked visually by an experimenter blind to treatment. An alternation is defined as entries into all three arms on consecutive occasions. The number of maximum alternations were therefore the total number of arm entries minus two and the percentage of alternation were calculated as (actual alternations / maximum alternations)×100. Calculated parameters consisted in the percentage of alternation (memory index) and the total number of arm entries (exploration index)(52-56).

Mice that showed an extreme behavior (Alternation percentage<20% or >90% or number of arm entries<8) were discarded. Animals were tested every week after the beginning of the treatment, at D15 D28; D30 and D38.

### Passive Avoidance Test (STPA)

All animals were tested for passive avoidance performance, an index of contextual long-term memory. The apparatus is a two-compartment (15×20×15 cm high) box with one illuminated with white polyvinylchloride walls and the other darkened with black polyvinylchloride walls and a grid floor. A guillotine door separates each compartment. A 60 W lamp positioned 40 cm above the apparatus lights up the white compartment during the experiment. Scrambled footshocks (0.3 mA for 3 s) can be delivered to the grid floor using a shock generator scrambler (MedAssociates, USA). The guillotine door was initially closed during the training session. Each mouse was placed into the white compartment. After 5 s, the door was raised. When the mouse was entered in the dark compartment and placed all its paws on the grid floor, the door was closed and the footshocks delivered for 3 s. The step-through latency, that is, the latency spent to enter the darkened compartment, and the number of vocalizations were recorded. The retention test was carried out 24 h after training. Each mouse was placed again into the white compartment. After 5 s, the door was raised. The step-through latency was recorded up to a cut-off time of 300 s (52-56).

Animals were tested every week after the beginning of the treatment, at D16/17 and D29/30 and D39/40.

### Statistical Analyses

All values were expressed as mean±S.E.M. Statistical analyses were performed on the different conditions using one-way ANOVA (F value), followed by the Dunnett's post-hoc multiple comparison test. Passive avoidance latencies do not follow a Gaussian distribution, since upper cut-off times are set. They were therefore analyzed using a Kruskal-Wallis non-parametric ANOVA (H value), followed by a Dunn's multiple comparison test. . . p<0.05 will be considered as statistically significant.

### Results

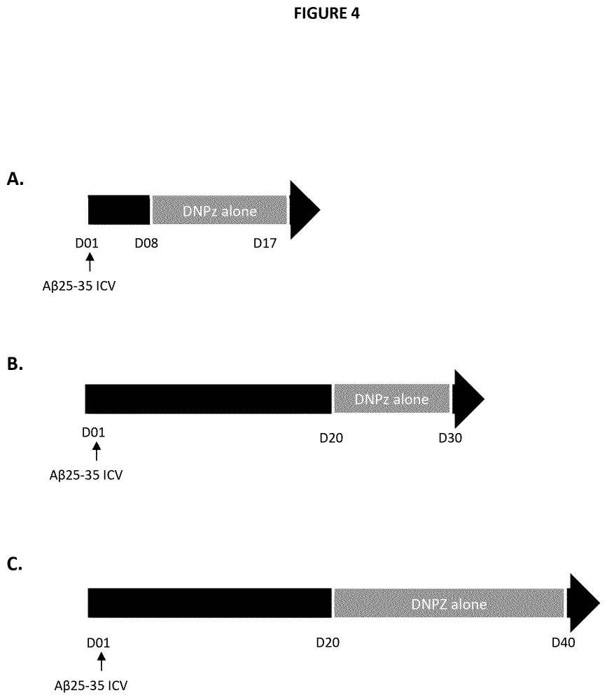

On day 1, all animals received an ICV injection either with Aβ or Sc.Aβ. The treatments with donepezil or with the vehicle started at D8 for 10 days; D20 for 11 (B) or 21 days (C) (FIG. 4).

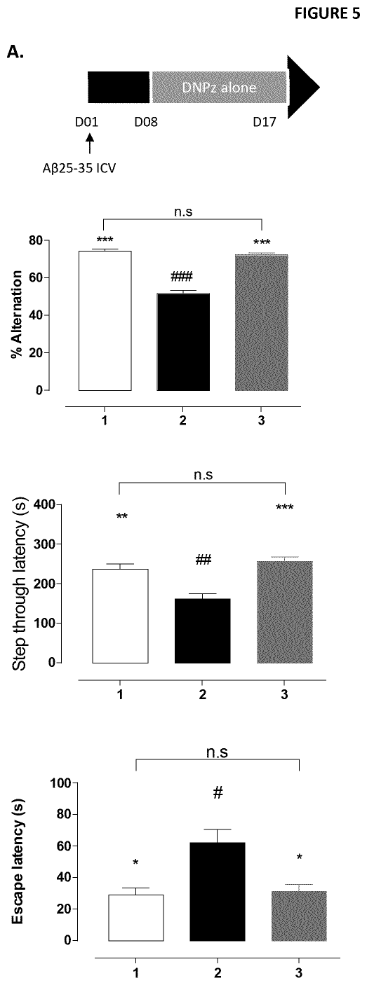

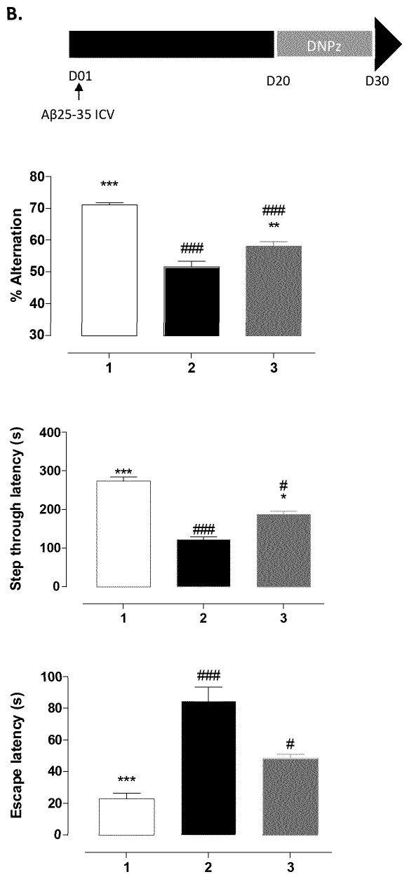

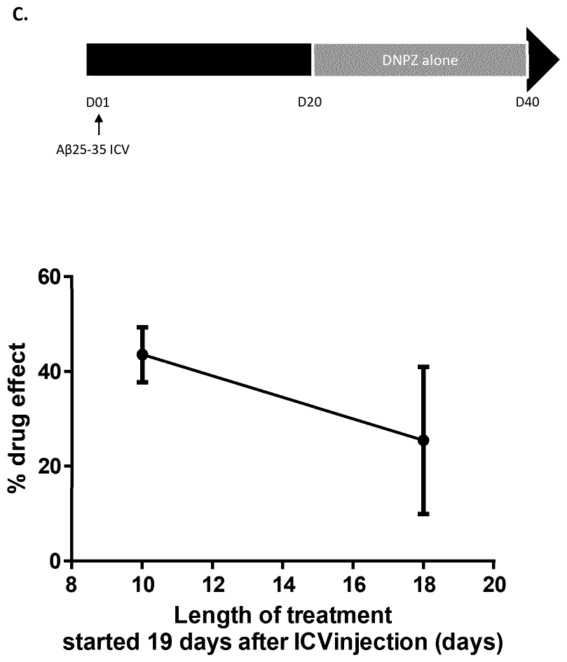

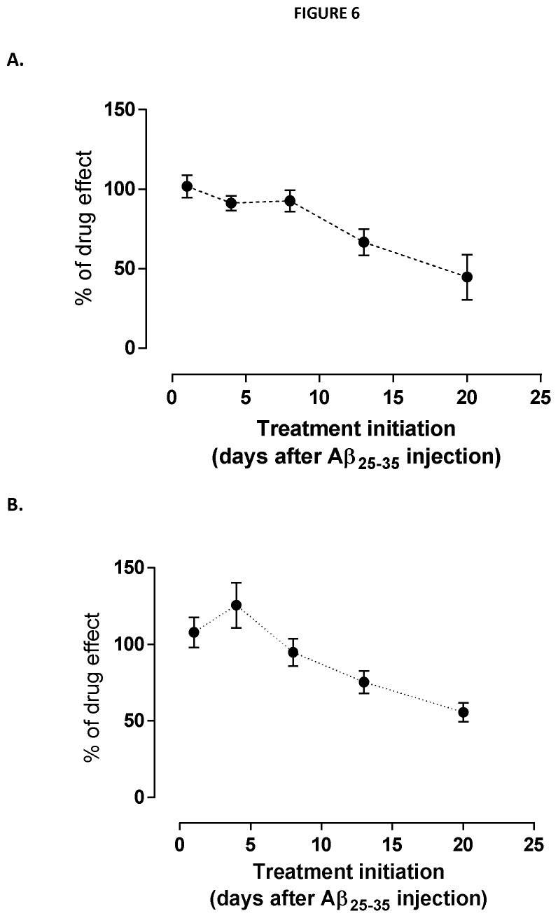

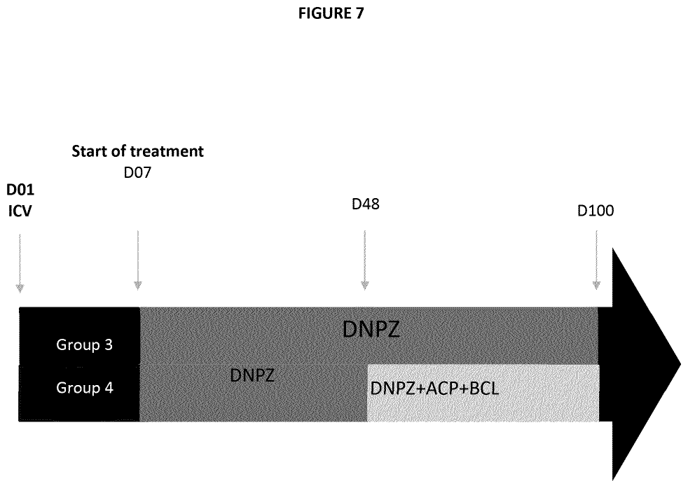

When the treatment started at D08 the dose of 1 mg/Kg of donepezil was able to fully recover Aβ₂₅₋₃₅-induced cognitive impairment assessed by Y-maze. Donepezil administered between D20 to D30 was partially active (44% of optimal activity) (FIG. 5A and B top graphs). Such a response is suboptimal.

The passive avoidance test is a readout of the fear conditioning memory and implicated also in long-term memory. It has been shown that one ICV injection of Aβ₂₅₋₃₅ is able to induce cognitive impairment compared to Sc.Aβ ICV injection. When the treatment started at D08 the dose of 1 mg/Kg of donepezil was able to fully recover Aβ₂₅₋₃₅-induced cognitive impairment assessed passive avoidance test. Donepezil administered between D20 to D30 was partially active (FIG. 5A and B bottom graphs).

In the last experiment, the treatment of Donepezil was initiated at D20 until D40 (21 days of treatment). The cognitive performances of animals were tested at D30 and D38 by Y-maze test. At D30, Donepezil effect (1 mg/Kg) was partially active (43% of activity—therefore further reproducing the abovementioned results following administration of donepezil between D20 to D30), and at D38 the drug effect was much lower, not statistically significant, only 25%. These data suggest that donepezil effect clearly decreases with time.

### Conclusion

This data demonstrated that donepezil at this therapeutic dose (1 mg/kg) was able to provide a full therapeutic effect. Indeed, when administered for eleven days starting 7 days after the induction of the pathology, this dose of donepezil resulted in a full recovery of the mice cognitive impairment induced by Aβ₂₅₋₃₅. When the initiation of the treatment was delayed, (D20), the response to donepezil was less optimal despite the effective therapeutic dose used. This design was able to mimic the loss of responsiveness of donepezil seen in the clinical context. As previously emphasized, studies with donepezil showed that the treatment improved the patient's cognitive function for the first 12 weeks, then the patient's cognitive function started declining to reach its baseline level only 30 weeks after initiation of treatment (20-23). The same limited efficacy was also described for rivastigmine (24) and galantamine (25). Therefore, it appears that patients lose their responsiveness to acetylcholinesterase inhibitors with time.

### C) Baclofen and Acamprosate Treatment of Diseases Related to Aβ Toxicity in an In Vivo Model That Lost Responsiveness To Therapeutic Effective Dose of Donepezil

### Protocol

### Animals

Male Swiss mice weighing 30-35 g, from JANVIER (Saint Berthevin, France), were housed and experiments were performed within AMYLGEN's animal facility (Direction Régionale de l′Alimentation, de l′Agriculture et de la Forêt du Languedoc-Roussillon, agreement #A 34-169-002 from May 02, 2014). Animals were housed in groups with access to food and water ad libitum, except during behavioral experiments. The temperature and humidity were controlled, and the animal facility on a 12 h/12 h light/dark cycle (lights off at 07:00 pm). Mice were numbered by marking their tail using permanent markers. All animal procedures were conducted in strict adherence to the European Union Directive of Sep. 22, 2010 (2010/63/UE).

Health of animals, general aspect of animals and activity were checked daily. Weight were monitored 3 times per week. Acute or delayed mortality were checked.

### Amyloid Peptide Injection (By ICV)

Male Swiss mice were anesthetized 5 minutes with isoflurane 2.5%, were restrained and the head immobilized, then the peptide was injected intracerebroventricularly (ICV) through a 28-gauge stainless-steel needle, 4 mm long. An injection volume of 3 μl was delivered gradually within 30 s and the needle was left in place for an additional 30 s before being removed (59). Animals were treated with amyloid peptide 25-35 (Aβ₂₅₋₃₅) peptide (9 nmol/mouse) or Scrambled Aβ peptide (Sc.Aβ) (9 nmol/mouse), in a final volume of 3 μl/mouse, according to the previously described method (52-56). Homogeneous preparation of the Aβ₂₅₋₃₅ peptide was performed according to the AMYLGEN's procedure.

### Treatment

All animals received a per os gavage using an inox steel cannula. All the treatments were administered under a volume calculated according to the individual body weight of each mouse (5 mL/Kg). Vehicle and donepezil (1 mg/Kg) administration were done once a day, and the mix acamprosate/baclofen twice a day (0.2 mg/Kg and 3 mg/Kg, respectively). Two groups of animals were administered with donepezil (1 mg/kg) from D7 until D100, the end of the study. In a first group of animals, only donepezil was administered during the whole study (group 3). In a second group of animals, at D48 when the activity of donepezil was lost as assessed by Y-maze, a combination of acamprosate and baclofen (0.2 mg/Kg and 3 mg/Kg respectively) was administered in addition of donepezil treatment (Group 4).

### Spontaneous Alternation Performance (Y-maze)

Mice were tested at D7, D14, D21, D28, D35, D42, D49, D56, D63, D70, D77, D91 and D98 for spontaneous alternation performance in the Y-maze, an index of spatial working memory. The Y-maze is designed according to Itoh et al.,1993 (57) and Hiramatsu and Inoue, 1999 (58), and is made of grey polyvinylchloride. Each arm is 40 cm long, 13 cm high, 3 cm wide at the bottom, 10 cm wide at the top, and converging at an equal angle. Each mouse is placed at the end of one arm and is allowed to move freely through the maze during an 8 min session. The series of arm entries, including possible returns into the same arm, are checked visually by an experimenter blind to treatment. An alternation is defined as entries into all three arms on consecutive occasions. The number of maximum alternations are therefore the total number of arm entries minus two and the percentage of alternation are calculated as (actual alternations / maximum alternations) x 100. Parameters are included the percentage of alternation (memory index) and total number of arm entries (exploration index) (52-56).

Mice that showed an extreme behavior (Alternation percentage<20% or >90% or number of arm entries<8) are discarded.

### Passive Avoidance Test (STPA)

At the end of the experiment (D99/D100), all animals were tested for passive avoidance performance, an index of contextual long-term memory. The apparatus is a two-compartment (15×20×15 cm high) box with one illuminated with white polyvinylchloride walls and the other darkened with black polyvinylchloride walls and a grid floor. A guillotine door separates each compartment. A 60 W lamp positioned 40 cm above the apparatus lights up the white compartment during the experiment. Scrambled footshocks (0.3 mA for 3 s) can be delivered to the grid floor using a shock generator scrambler (MedAssociates, USA). The guillotine door is initially closed during the training session. Each mouse is placed into the white compartment. After 5 s, the door is raised. When the mouse enters in the darkened compartment and places all its paws on the grid floor, the door closes and the footshocks delivers for 3 s. The step-through latency, that is, the latency spent to enter the darkened compartment, and the number of vocalizations is recorded. The retention test is carried out 24 h after training. Each mouse is placed again into the white compartment. After 5 s, the door is raised. The step-through latency is recorded up to a cut-off time of 300 s (52-56).

### Statistical Analyses

All values are expressed as mean±S.E.M. Statistical analyses are performed on the different conditions using one-way ANOVA (F value), followed by the Dunnett's post-hoc multiple comparison test. Passive avoidance latencies do not follow a Gaussian distribution, since upper cut-off times are set. They are therefore analyzed using a Kruskal-Wallis non-parametric ANOVA (H value), followed by a Dunn's multiple comparison test. p<0.05 will be considered as statistically significant.

### Results

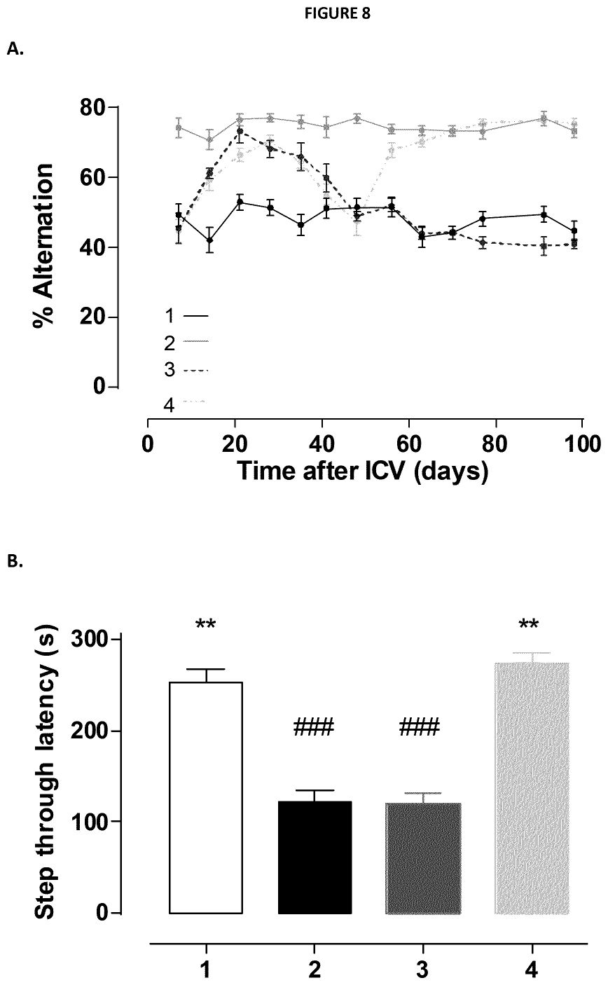

DNPz treatment initiated at D7 showed a maximal effect between D21 and D28. This effect was not significantly different from Sc.Aβ (70 to73% of alternation for DNPz treated animals, 76% of alternation for Sc.Aβ-vehicle treated animals) (FIG. 8A). At D42, DNPz efficacy decreased, and cognitive performances of animals became similar to those of Aβ₂₅₋₃₅ injected animals treated with the vehicle (47% of alternation for Aβ₂₅₋₃₅-DNPz treated animals against 51% of alternation for Aβ₂₅₋₃₅-vehicle treated animals) (FIG. 8A). After D48, donepezil inactivity was sustained until D100 in the group of animals treated only with donepezil, and their cognitive impairment was similar to that of animals injected with Aβ₂₅₋₃₅ and treated with the vehicle.

At D48, when the activity of donepezil (1 mg/kg) was lost as assessed by Y-maze, one group of animals (Group 4) was supplemented at D49 until D100 with a combination of acamprosate and baclofen (0.2 mg/Kg and 3 mg/Kg respectively).

One week after supplementation with the combination of acamprosate and baclofen, the animals displayed a significant improvement of cognitive performances. In fact, at D56, no statistical difference was observed between animals supplemented with a combination acamprosate and baclofen and those injected with the scrambled Aβ peptides. The full recovery was reached only two weeks after initiation of the supplementation with the combination of acamprosate and baclofen (FIG. 8A). In the same manner, at the end of the experiment (D99/D100), fear conditioning memory assessed by STPA was altered for the animals treated only with donepezil (FIG. 8B). By contrast, in the group of animals supplemented with the combination of acamprosate and baclofen, cognitive impairments was fully restored (274 s of STL vs 251 secs for Sc.Aβ vehicle treated) (FIG. 8B).

This data demonstrated that the addition of a combination of acamprosate and baclofen after the loss of donepezil efficacy fully and quickly restored cognitive performances as spatial working memory and long-term memory in mice model of cognitive impairment induced by Aβ₂₅₋₃₅.

### Conclusion

This data further confirmed that a therapeutic effective dose of donepezil resulted in an improvement, positive significant effect, of the mice cognitive impairment in a mice model of Alzheimer's disease.

It also further demonstrated that this effect then diminished with the mice becoming non-responsive to the therapeutic effective dose of donepezil.

Most importantly, it demonstrated that further treating the mice at this stage with a combination of acamprosate and baclofen resulted in full recovery of the mice cognitive functions. The full recovery was achieved only two weeks after administration of the combination of acamprosate and baclofen.

This result is especially surprising considering that in the same model of Aβ₂₅₋₃₅-induced cognitive impairment, the sole administration of a combination of baclofen and acamprosate at the same dose, for a period of 11 days (D20 to D30) and up to 21-23 days (D20 to D40, D41/D42), alleviated cognitive impairments of the mice but was not able to fully rescue it (see FIGS. 2D and 3D).

### REFERENCES

1. Crook R, Verkkoniemi A, Perez-Tur J, Mehta N, Baker M, Houlden H, Farrer M, Hutton M, Lincoln S, Hardy J, Gwinn K, Somer M, Paetau A, Kalimo H, Ylikoski R, Pöyhönen M, Kucera S & Haltia M (1998) A variant of Alzheimer's disease with spastic paraparesis and unusual plaques due to deletion of exon 9 of presenilin 1. Nat. Med. 4, 452-5.
2. Houlden H, Baker M, McGowan E, Lewis P, Hutton M, Crook R, Wood N W, Kumar-Singh S, Geddes J, Swash M, Scaravilli F, Holton J L, Lashley T, Tomita T, Hashimoto T, Verkkoniemi A, Kalimo H, Somer M, Paetau A, Martin J J, Van Broeckhoven C, Golde T, Hardy J, Haltia M & Revesz T (2000) Variant Alzheimer's disease with spastic paraparesis and cotton wool plaques is caused by PS-1 mutations that lead to exceptionally high amyloid-beta concentrations. Ann. Neurol. 48, 806-8.
3. Kwok J B, Taddei K, Hallupp M, Fisher C, Brooks W S, Broe G A, Hardy J, Fulham M J, Nicholson G A, Stell R, St George Hyslop P H, Fraser P E, Kakulas B, Clarnette R, Relkin N, Gandy S E, Schofield P R & Martins R N (1997) Two novel (M233T and R278T) presenilin-1 mutations in early-onset Alzheimer's disease pedigrees and preliminary evidence for association of presenilin-1 mutations with a novel phenotype. Neuroreport 8, 1537-42.
4. Verkkoniemi A, Kalimo H, Paetau A, Somer M, Iwatsubo T, Hardy J & Haltia M (2001) Variant Alzheimer disease with spastic paraparesis: neuropathological phenotype. J. Neuropathol. Exp. Neurol. 60, 483-92.
5. Citron M (2004) Strategies for disease modification in Alzheimer's disease. Nat. Rev. Neurosci. 5, 677-85.
6. Suh Y-H & Checler F (2002) Amyloid precursor protein, presenilins, and alpha-synuclein: molecular pathogenesis and pharmacological applications in Alzheimer's disease. Pharmacol. Rev. 54, 469-525.
7. Blacker D, Albert M S, Bassett S S, Go R C, Harrell L E & Folstein M F (1994) Reliability and validity of NINCDS-ADRDA criteria for Alzheimer's disease. The National Institute of Mental Health Genetics Initiative. Arch. Neurol. 51, 1198-204.
8. Rossor M N, Fox N C, Freeborough P A & Harvey R J (1996) Clinical features of sporadic and familial Alzheimer's disease. Neurodegeneration 5, 393-7.
9. Glenner G G, Wong C W, Quaranta V & Eanes E D (1984) The amyloid deposits in Alzheimer's disease: their nature and pathogenesis. Appl. Pathol. 2, 357-69.
10. Ballatore C, Lee VM-Y & Trojanowski J Q (2007) Tau-mediated neurodegeneration in Alzheimer's disease and related disorders. Nat. Rev. Neurosci. 8, 663-72.
11. DiLuca M, Bell K F S & Claudio Cuello A (2006) Altered synaptic function in Alzheimer's disease. Eur. J. Pharmacol. 545, 11-21.
12. Hardy J A & Higgins G A (1992) Alzheimer's disease: the amyloid cascade hypothesis. Science 256, 184-5.
13. Braak H & Braak E (1991) Neuropathological stageing of Alzheimer-related changes. Acta Neuropathol. 82, 239-59.
14. Maccioni R B, Farias G, Morales I, Navarrete L (2010 April) The revitalized tau hypothesis on Alzheimer's disease. Arch Med Res. 41(3): 226-31.
15. Golde T E (2005) The Abeta hypothesis: leading us to rationally-designed therapeutic strategies for the treatment or prevention of Alzheimer disease. Brain Pathol. 15, 84-7.
16. Hardy J & Selkoe D J (2002) The amyloid hypothesis of Alzheimer's disease: progress and problems on the road to therapeutics. Science 297, 353-6.
17. Selkoe D J (2000) The genetics and molecular pathology of Alzheimer's disease: roles of amyloid and the presenilins. Neurol. Clin. 18, 903-22.
18. Zlokovic B V (2008) The blood-brain barrier in health and chronic neurodegenerative disorders. Neuron 57, 178-201.
19. Budd Haeberlein S L & Lipton S A (2009) Excitotoxicity in neurodegenerative disease. In Encyclopedia of neuroscience (Squire L R, ed), pp. 77-86. Elsevier.
20. Burns A, Rossor M, Hecker J, Gauthier S, Petit H, Moller H J, Rogers S L, Friedhoff L T and the International Donepezil Study Group. The Effects of Donepezil in Alzheimer's Disease—Results from a Multinational Trial. Dement Geriatr Cogn Disord 1999; 10:237-244.
21. Rogers S L, Farlow M R, Doody R S, Mohs R, Friedhoff L T, and the Donepezil Study Group. A 24-week, double -blind, placebo controlled trial of donepezil in patients with Alzheimer's disease. Neurology 50, January 1998.
22. Rogers S L, Doody R S, Pratt R D, Ieni J R. Long-term efficacy and safety of donepezil in the treatment of Alzheimer's disease: a final analysis of a US multicenter open-label study. European Neuropsychopharmacology 10 (2000) 195-203.
23. Rocca P, Cocuzza E, Marchiaro L, Bogetto F. Donepezil in the treatment of Alzheimer's disease Long-term efficacy and safety. Progress in Neuro-Psychopharmacology & Biological Psychiatry 26 (2002) 369-373.
24. Feldman H H, Lane R. Rivastigmine: a placebo controlled trial of twice daily and three times daily regimens in patients with Alzheimer's disease. J Neurol Neurosurg Psychiatry 2007; 78: 1056-1063.
25. Raskind M A. Update on Alzheimer Drugs (Galantamine). The Neurologist, Vol 9 Number 5, September 2003.
26. Rosen W G, Mohs R C, Davis K L. A new rating scale for Alzheimer's disease. Am J Psychiatry 1984;141:1356-64.
27. Folstein M F, Folstein S E, McHugh P R. “Mini-mental state”. A practical method for grading the cognitive state of patients for the clinician. J Psychiatr Res 1975;12:189-98.
28. Hughes C P, Berg L, Danziger W L, Coben L A, Martin R L. A new clinical scale for the staging of dementia. Br J Psychiatry. 1982;140:566-572.
29. Hughes C P. A new clinical scale for the staging of dementia. Br J Psychiatry. 1982 June;140: 566-72.
30. Knopman D S. The Clinician Interview-Based Impression (CIBI): a clinician's global change rating scale in Alzheimer's disease. Neurology. 1994 December;44(12):2315-21.
31. Teunisse S. The interview for deterioration in daily living activities in dementia: agreement between primary and secondary caregivers. Int Psychogeriatr. 1997;9 Suppl 1:155-62.
32. Lawton. Assessment of Older People: Self-Maintaining and Instrumental Activities of Daily Living. Gerontologist 1969 ; 9 (3):179-86.
33. Isaacs B, Kennie A. The Set test as an aid to the detection of dementia in old people. The British Journal of Psychiatry, 1973, 123, 467-470.
34. Jinping Wang. ADCOMS: a composite clinical outcome for prodromal Alzheimer's disease trials. J Neurol Neurosurg Psychiatry. 2016 September; 87(9): 993-999.
35. Stella V J (2007) Prodrugs: challenges and rewards. (A. Press and Springer, eds). Springer Singapore Pte. Limited, New-York.
36. Wermuth C G (2011) The Practice of Medicinal Chemistry. Elsevier Science.
37. Pezron I, Mitra A K, Duvvuri S & Tirucherai G S (2002) Prodrug strategies in nasal drug delivery. Expert Opin. Ther. Pat. 12, 331-340.
38. Stella V J (2004) Prodrugs as therapeutics. Expert Opin. Ther. Pat. 14, 277-280.
39. Stella V J & Nti-Addae K W (2007) Prodrug strategies to overcome poor water solubility. Adv. Drug Deliv. Rev. 59, 677-94.
40. Beaumont K, Webster R, Gardner I & Dack K (2003) Design of ester prodrugs to enhance oral absorption of poorly permeable compounds: challenges to the discovery scientist. Curr. Drug Metab. 4, 461-85.
41. Higuchi T & Stella V J (1975) Pro-drugs as Novel Drug Delivery System, ACS Sympos American Chemical Society, Washington, D.C.
42. Roche E B (1977) Design of biopharmaceutical properties through prodrugs and analogs: a symposium, American P The Academy, Washington, D.C.
43. Lal R, Sukbuntherng J, Tai E H L, Upadhyay S, Yao F, Warren M S, Luo W, Bu L, Nguyen S, Zamora J, Peng G, Dias T, Bao Y, Ludwikow M, Phan T, Scheuerman R A, Yan H, Gao M, Wu Q Q, Annamalai T, Raillard S P, Koller K, Gallop M A & Cundy K C (2009) Arbaclofen placarbil, a novel R-baclofen prodrug: improved absorption, distribution, metabolism, and elimination properties compared with R-baclofen. J. Pharmacol. Exp. Ther. 330, 911-21.
44. Xu F, Peng G, Phan T, Dilip U, Chen J L, Chernov-Rogan T, Zhang X, Grindstaff K, Annamalai T, Koller K, Gallop M A & Wustrow D J (2011) Discovery of a novel potent GABA(B) receptor agonist. Bioorg. Med. Chem. Lett. 21, 6582-5.
45. Wishart D S, Knox C, Guo A C, Cheng D, Shrivastava S, Tzur D, Gautam B & Hassanali M (2008) DrugBank: a knowledgebase for drugs, drug actions and drug targets. Nucleic Acids Res. 36, D901-6.
46. Leach A R & Gillet V J An Introduction to Chemoinformatics (Springer-Verlag New York Inc, ed.).
47. Rahman S A, Bashton M, Holliday G L, Schrader R & Thornton J M (2009) Small Molecule Subgraph Detector (SMSD) toolkit. J. Cheminform. 1, 12.
48. Stahl H & Wermuth C G (2011) Pharmaceutical salts: Properties, selection, and use, 2nd ed. (Wiley-VCH, ed.).
49. Hanafi R, Mosad S, Abouzid K, Niess R & Spahn-Langguth H (2011) Baclofen ester and carbamate prodrug candidates: a simultaneous chromatographic assay, resolution optimized with DryLab. J. Pharm. Biomed. Anal. 56, 569-76.
50. Gennaro A R (2000) Remington: The Science and Practice of Pharmacy, 20th ed. (A. D. Gennaro, W. Lippincott, and Wilkins, eds.) Lippincott Williams & Wilkins.
51. Swarbrick J & Boylan J C (eds.) Encyclopedia of Pharmaceutical Technology Dekker, Marcel, New-York.
52. Maurice T, Lockhart B P, Privat A. Amnesia induced in mice by centrally administered β-amyloid peptides involves cholinergic dysfunction. Brain Res 706, 181-193, 1996.
53. Maurice T, Su T P, Privat A. Sigma₁ (σ₁) receptor agonists and neurosteroids attenuate β₂₅₋₃₅-amyloid peptide-induced amnesia in mice through a common mechanism. Neuroscience, 83, 413-428, 1998.
54. Meunier J, Ieni J, Maurice T. The anti-amnesic and neuroprotective effects of donepezil against amyloid β₂₅₋₃₅ peptide-induced toxicity in mice involve an interaction with the σ₁ receptor. Br J Pharmacol, 149, 998-1012, 2006.
55. Villard V, Espallergues J, Keller E, Alkam T, Nitta A, Yamada K, Nabeshima T, Vamvakides A, Maurice T. Anti-amnesic and neuroprotective effects of the aminotetrahydrofuran derivative ANAVEX1-41 against amyloid β₂₅₋₃₅-induced toxicity in mice. Neuropsychopharmacology, 34, 1552-66, 2009.
56. Villard V, Espallergues J, Keller E, Vamvakides A, Maurice T. Anti-amnesic and neuroprotective potentials of the mixed muscarinic receptor/sigma₁ (σ₁) ligand ANAVEX2-73, a novel aminotetrahydrofuran derivative. J Psychopharmacol, 25, 1101-17, 2011.
57. Itoh, J, Ukai, M & Kameyama, T Dynorphin A-(1-13) markedly improves scopolamine-induced impairment of spontaneous alternation performance in mice. European journal of pharmacology, 236, 341-345, 1993.
58. Hiramatsu, M & Inoue, K. Nociceptin/orphanin FQ and nocistatin on learning and memory impairment induced by scopolamine in mice. British journal of pharmacology, 127, 655-660, 1999.
59. Haley, T J & McCormick, W G. Pharmacological effects produced by intracerebral injection of drugs in the conscious mouse. British journal of pharmacology and chemotherapy, 12, 12-15, 1957.

1-21. (canceled)

22. A method of treating a subject having Alzheimer's disease or an Alzheimer's disease related disorder that is not responding to an inhibitor of acetylcholinesterase comprising administering baclofen and acamprosate, or pharmaceutically acceptable salts or derivatives thereof or a composition thereof to the subject having Alzheimer's disease or an Alzheimer's disease related disorder that is not responding to an inhibitor of acetylcholinesterase.

23. The method according to claim 22, wherein the subject is not-responding to said inhibitor of acetylcholinesterase when his/her performance in a cognitive test after treatment with said inhibitor is suboptimal or the subject is not-responding to said inhibitor of acetylcholinesterase when his/her performance in a cognitive test is not improved by said inhibitor.

24. The method according to claim 23, wherein the cognitive test is selected from ADAS-Cog, MMSE and CDR-SB.

25. The method according to claim 22, wherein the subject is a patient under treatment with therapeutic doses of said inhibitor of acetylcholinesterase and who has lost optimal responsiveness to said inhibitor.

26. The method according to claim 22, wherein the subject is a patient who has been under treatment with the inhibitor of acetylcholinesterase for a period of at least 12 weeks.

27. The method according to claim 26, wherein the subject is a patient who has been under treatment with the inhibitor of acetylcholinesterase for a period of at least 6 months.

28. The method according to claim 22, wherein said inhibitor of acetylcholinesterase is selected from the group consisting of donepezil, rivastigmine and galantamine.

29. The method according to claim 28, wherein said inhibitor of acetylcholinesterase is donepezil.

30. The method according to claim 22, the method comprising the administration of said inhibitor of acetylcholinesterase.

31. The method according to claim 30, wherein said inhibitor is donepezil at a dose between 1 and 20 mg per day.

32. The method according to claim 30, wherein said inhibitor is rivastigmine at a dose between 1 and 30 mg per day.

33. The method according to claim 30, wherein said inhibitor is galantamine at a dose between 8 and 40 mg per day.

34. The method according to claim 22, wherein baclofen and acamprosate are the only active agent.

35. The method according to claim 22, wherein the composition comprises a pharmaceutically acceptable carrier or excipient.

36. The method according to claim 22, wherein the compounds in said composition are formulated or administered together, separately or sequentially.

37. The method according to claim 22, wherein the ratio of acamprosate/baclofen (W:W) is between 0.05 and 1000.

38. The method according to claim 22, wherein the dose of baclofen is less than 100 mg/day.

39. The method according to claim 22, wherein the dose of acamprosate is less than 1000 mg/day.

40. A method of treating a subject having Alzheimer's disease or an Alzheimer's disease related disorder under treatment with an inhibitor of acetylcholinesterase comprising the administration of a composition comprising baclofen and acamprosate, or pharmaceutically acceptable salts or derivatives thereof, wherein said composition is administered to the subject when the subject has lost responsiveness to said inhibitor of acetylcholinesterase.
